(19) United States
(12) Patent Application Publication
(10) Pub. No.: US 2024/0153636 A1
(43) Pub. Date: May 9, 2024

(54) MODELS FOR CANCER DISEASES

(71) Applicant: UNIVERSITY OF SOUTH FLORIDA, Tampa, FL (US)

(72) Inventors: Chris Peter Tsokos, Tampa, FL (US); Aditya Chakraborty, Tampa, FL (US)

(21) Appl. No.: 18/487,984

(22) Filed: Oct. 16, 2023

Related U.S. Application Data
(60) Provisional application No. 63/416,414, filed on Oct. 14, 2022.

(51) Int. Cl. G16H 50/20; G16H 50/50; G16H 50/70

(52) U.S. Cl. CPC G16H 50/20; G16H 50/50; G16H 50/70

(57) ABSTRACT

Methods and systems for cancer disease estimation are disclosed. The methods and systems include: receiving an input for a patient for selecting at least one of: a cancer disease deep learning model, a cancer disease stochastic model, or a cancer disease probability distribution function-based model; and perform cancer disease estimation processes based on the cancer disease deep learning model, the cancer disease stochastic model, or the cancer disease probability distribution function-based model. Other aspects, embodiments, and features are also claimed and described.

### CROSS-REFERENCE TO RELATED APPLICATION(S)

This application claims the benefit of U.S. Provisional Patent Application Ser. No. 63/416,414, filed Oct. 14, 2022, the disclosure of which is hereby incorporated by reference in its entirety, including all figures, tables, and drawings.

### STATEMENT REGARDING FEDERALLY SPONSORED RESEARCH

N/A

### BACKGROUND

Cancer is a leading cause of death worldwide. After the detection of cancer, doctors usually perform some additional tests to understand better if cancer has been spread or the locations of spreading areas of the cancer. Imaging tests, such as a PET scan, help doctors identify the presence of cancerous growths. With these tests, doctors try to establish cancer's stage of a given patient with cancer. Staging helps explicate the advancement of cancer. It also assists doctors in deciding treatment options. Once a diagnosis has been made, the doctor allocates the patient a stage based on the test results. Once a patient is diagnosed with cancer, he/she or his/her family members would be interested in knowing how long the expected/predicted survival would be. This question is usually asked by patients with a terminal illness to their doctors. However, it is hard to provide the exact answer to these questions because doctors provide an answer which is mainly subjective. What are needed are systems and methods that address one or more of these shortcomings.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects of the present disclosure, methods, systems, and apparatus for cancer disease estimation are disclosed. These methods, systems, and apparatus can include steps or components for: receiving an input for selecting at least one of: a cancer disease deep learning model, a cancer disease stochastic model, or a cancer disease probability distribution function-based model. In response to the input to select the cancer disease deep learning model, these methods, systems, and apparatus can further include steps or components for: obtaining a trained cancer disease deep learning model; receiving a plurality of entries corresponding to a plurality of risk factors for a patient; providing the plurality of entries to the trained cancer disease deep learning model; and providing a first result for the patient based on the trained cancer disease deep learning model. In response to the input to select the cancer disease stochastic model, these methods, systems, and apparatus can further include steps or components for: receiving a plurality of patient inputs for the patient; receiving a treatment option; determining a survival monitoring indicator for the patient based on the plurality of patient inputs and the treatment option; and producing a second result based on the treatment option and the survival monitoring indication. In response to the input to select the cancer disease probability distribution function-based model, these methods, systems, and apparatus can further include steps or components for: receiving a survival time input for the patient; determining three parameters; determining a parametric survival function for the patient; and providing a third result based on the parametric survival function and the survival time input.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 shows pancreatic cancer data with relevant risk factors according to some embodiments.

FIG. 2 illustrates an example process for developing an example deep learning analytical model according to some embodiments.

FIGS. 3A and 3B illustrate graphs of RMSE and MAE of the example deep learning model according to some embodiments.

FIG. 4 shows relative importance of risk factors used in the example model according to some embodiments.

FIG. 5 illustrates an example data driven analytical process according to some embodiments.

FIG. 6 is a graph showing stochastic growth intensity functions (SGIFs) for a group with two different treatments according to some embodiments.

FIG. 7 is a graph showing stochastic growth intensity functions (SGIFs) for a group with two different treatments according to some embodiments.

FIG. 8 shows pancreatic cancer data sorted by gender and stages according to some embodiments.

FIG. 9 illustrates the probability density function (pdf) and cumulative distribution function (cdf) of the patients in stage I according to some embodiments.

FIG. 10 shows the histogram, pdf and cdf plots of stage II pancreatic cancer patients according to some embodiments.

FIG. 11 shows the histogram, pdf and cdf plots of stage III pancreatic cancer patients according to some embodiments.

FIG. 12 describes the histogram, pdf, and cdf of male survival time respectively in stage IV according to some embodiments.

FIG. 13 describes the histogram, pdf, and cdf of female survival time respectively in stage IV according to some embodiments.

FIG. 14 shows a parametric survival plot of pancreatic cancer patients in Stage I according to some embodiments.

FIG. 15 shows a parametric survival plot of pancreatic cancer patients in Stage II according to some embodiments.

FIG. 16 shows a parametric survival plot of pancreatic cancer patients in Stage III according to some embodiments.

FIG. 17 shows a parametric survival plot of male pancreatic cancer patients in Stage IV according to some embodiments.

FIG. 18 shows a parametric survival plot of female pancreatic cancer patients in Stage IV according to some embodiments.

FIG. 19 shows testing for difference in survival times across gender according to some embodiments.

FIG. 20 shows a histogram and probability density of survival times of combined pancreatic cancer patients according to some embodiments.

FIG. 21 shows a cdf plot for the survival times of overall pancreatic cancer patients according to some embodiments.

FIG. 22 shows a parametric survival plot of overall pancreatic cancer patients according to some embodiments.

FIG. 23 is a block diagram conceptually illustrating a hardware system to implement processes in FIG. 24 and/or 25 according to some embodiments.

FIG. 24 is a flow diagram illustrating an example process for cancer disease estimation according to some embodiments.

FIG. 25 is a flow diagram illustrating an example process for cancer disease deep learning model training according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

Pancreatic adenocarcinoma is one of the deadliest carcinogenic diseases affecting people worldwide. Early detection of pancreatic adenocarcinoma is rare, and once diagnosed in later stages, the chances of survival are extremely low. The present disclosure includes three parts.

First, the present disclosure includes an artificial intelligence AI-driven analytical model to predict the survival times of individual pancreatic patients using extreme gradient boosting. The AI-driven analytical model can identify ten risk factors that contribute significantly to the survival of the patient diagnosed with pancreatic adenocarcinoma. Once these risk factors are identified, the system can rank them with respect to the percentage of contribution to pancreatic cancer. For example, the top three most contributing risk factors of pancreatic adenocarcinoma are the age of the patient (35.5%), current body mass index (BMI) (24.3%), and the number of years smoking cigarette (14.93%). The predictive analytical model is 96.42% accurate. This model has been statistically tested to give excellent predictions.

Second, the disclosure further includes a stochastic model that is a function of a Survival Intensity Function (SIF) and a Survival Monitor Indicator (SMI). The SIF can identify the survival rate of pancreatic cancer patients as a function of time, and SMI can monitor the behavior of pancreatic cancer patients at a specific time. The SMI is an important decision-making indicator that conveys three conditions (the patients' survival time is increasing, the patients' survival time remains the same, and the patients' survival time is decreasing) of the pancreatic cancer patients at a specific time. The SMI offers a number of important uses on the subject matter. For example, in the case of pancreatic cancer patients, they have three different treatments (1) Chemotherapy only (C), 2) Radiation only (R), and 3) Chemotherapy and Radiation both (C+R)). The SMI can be used to evaluate the effectiveness of the administered treatment to a given patient. That is, if the treatment worsens the patient's cancer, the treatment has no effect on cancer, or the treatment is effective on the cancer. To inventors' knowledge, there is no such analytical model that offers this important evaluation of different treatments. The flexibility of the described model lies in the fact that it can incorporate any number of additional treatments. The survival Intensity Function (SIF) offers an analytical and graphical display of the survival rate of change of pancreatic cancer patients as a function of time. This information is important to scientists in monitoring the subject matter for making important decisions on how to proceed. Furthermore, our innovation categories pancreatic cancer patients from three race groups, Caucasian, African American, and other in utilizing the proposed analytical model. In addition, our analysis is performed at four different stages of pancreatic cancer and three different age groups, 40 to 59, 60 to 79, and 80 and older.

Third, the disclosure further includes a generalized Pareto probability distribution function. Based on this statistical analysis, the survival times of pancreatic cancer patients can be characterized and a more accurate measurement/estimation of the survival analysis of the subject patients can be obtained. That is, it can provide more accurate results than the classical methods that are commonly used. This analysis can be used for multiple purposes, such as to provide information to patients and their families to help them understand what to expect; to provide information to healthcare providers to assist in determination of when treatments are warranted/no longer warranted; and the like.

Systems and methods of the present disclosure may also be adapted to monitor (e.g., on a periodic basis, in real time, or as new information is available) the relationship between a given pancreatic cancer drug, or other therapy or intervention, and the expected lifespan of patients receiving the same. For example, a system may monitor a patient's electronic medical record or the data repository associated with a clinical trial or other study, and can indicate patients, doctors, or both whether the drug, therapy, or intervention is likely proving effective or not for a given patient or population. For example, currently-known pancreatic cancer treatment drugs include Lynparza (also known as Olaparib), Pembrolizumab (also known as Keytruda), Atezolizumab (also known as Tencentzig), and Olaparib. However, these drugs are not known to be equally effective, or effective at all, for given patients. Thus, as the drugs are being used in treatment, or being studied in a clinical setting, a system of the present disclosure can provide information to a portal, dashboard, or other user interface indicating the likelihood that the drug is extending life expectancy, having no effect, or having a detrimental effect. Additionally, this method may be used in clinical trials by pharmaceutical companies which are constantly working to develop new drugs and treatments.

Specifically, embodiments may utilize a Survival Intensity Function (SIF) and Survival Monitor Indicator (SMI) used to determine whether a given drug, therapy, or intervention is effective, if no effect occurs, or if the drug is counter productive (e.g., it hurts the patient's progress). The SMI can identify if the specific pancreatic drug is effective or not. Specifically, an SMI value of less than 1 indicates that the patient's survival time increases, and the treatment is effective. Moreover, an SMI value equal to 1 indicates that the patient's survival time remains the same, and the treatment has no effect. Finally, an SMI value greater than 1 indicates the survival time of the patient is decreasing, and the therapy is hurting the patient.

### Example 1: A Real Data-Driven Analytical Predictive Model for Pancreatic Cancer Patients Using Extreme Gradient Boosting

This section describes building an efficient survival model based on the risk factors and identify the most contributing factors influencing the survival times of patients diagnosed with cancer diseases (e.g., pancreatic cancer). In this disclosure, the inventors developed a real data-driven machine learning predictive model with 800 pancreatic cancer patients' information and ten risk factors to predict their survival times. To check the validity of the model, the inventors compared the model's performance with ten deep neural network models, grown sequentially with different activation functions and optimizers. The described XGBoost model outperformed all competing models with regards to root mean square error (RMSE). After developing the model, all the individual risk factors were ranked according to their individual contribution to the response predictions, which can be considered for pancreatic research organizations to spend their resources on the risk factors causing/influencing the particular type of cancer. The three most influencing risk factors affecting the survival of pancreatic cancer patients are found to be the age of the patient, current BMI, and cigarette smoking years with contributing percentages 35.5%, 24.3%, and 14.93%, respectively. The described predictive model is approximately 96.42% accurate in predicting the survival times of the patients diagnosed with pancreatic cancer and performs excellently on test data. The analytical model can be implemented for prediction purposes for the survival times of pancreatic cancer patients, given a set of risk factors.

The response variable can be the survival time (in years). Although in most cases, pancreatic cancer remains incurable, the example model in the present disclosure can accurately predict the survival rates of individuals with pancreatic cancer.

Data Description: The data for the example model has been obtained from National Cancer Institute (NIH). The data contains information on patients diagnosed with pancreatic adenocarcinoma. The inventors treated the survival time (in years) as the response in developing the example model and considered cause-specific death (deaths due to pancreatic cancer) for each patient. Patient survival time is one of the factors in all cancer studies. Based on the assessed severity of cancer, the prognosis can be determined, and the treatment options can be found. There was a total of 800 patient information to develop the example model after eliminating the missing observations for which several risk factors were missing. the response variable is the survival time of patients. There are a total of ten risk factors used in the predictive analysis. Seven of those are categorical in nature, and three of them are numeric variables. The descriptions of the risk factors are as follows.

1. panc_exitage (Numeric) (X₁): Age of diagnosis of the patient. 2. Stage (Categorical) (X₂): Pancreatic Cancer Stages, categorized as a) localized, b) regional, and c) distant. 3. asp (Categorical) (X₃): Does the person use Aspirin Regularly? 4. ibup (Categorical) (X₄): Does the person use Ibuprofen Regularly? 5. fh_Cancer (Categorical) (X₅): The number of first-degree relatives with any type of cancer. 6. Sex (Categorical) (X₆): Sex of the individual. 7. BMI (numeric) (X₇): Current Body Mass Index (BMI) at Baseline (In lb/in2). 8. Cigarette Years (numeric) (X₈): The total number of years the patient smoked. 9. gallblad_f (Categorical) (X₉): Did the individual ever have gall bladder stones or inflammation? 10. hyperten_f (Categorical) (X₁₀): Did the individual ever have high blood pressure?

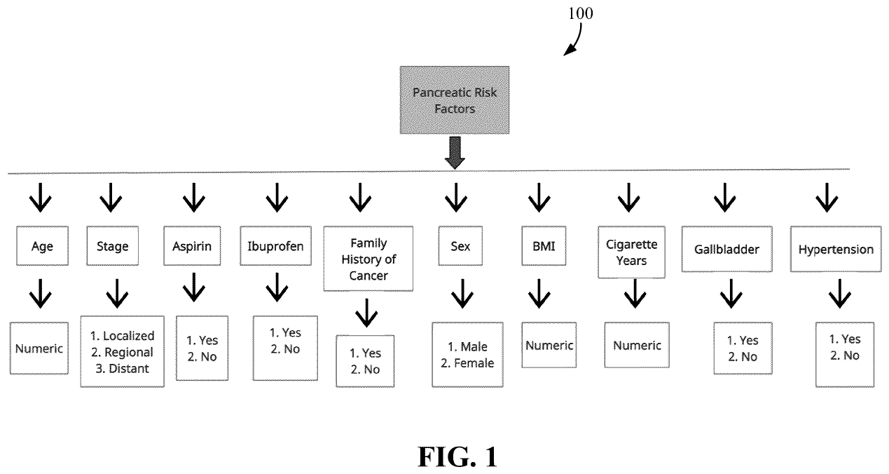

A schematic diagram of the data used in the present disclosure with the description of risk factors is shown in FIG. 1. As shown in FIG. 1, seven out of ten risk factors are categorical, having two or more categories. In the data, the inventors obtained a p-value of 0.47 (>0.05), which suggests that there is no statistically significant difference between the true mean survival times of patients from both genders at 5% level of significance. Therefore, the analysis combines the information of males and females.

Analytical Modeling and Results: In some examples, the example model can predict the survival times of cancer patients (e.g., pancreatic cancer patients) with the highest degree of accuracy. For that, a number of machine learning (ML) and deep learning (DL) models have been tested and validated on the data. The inventors used Feed forward Deep Learning Models with different layers, optimizer, and activation functions. The deep learning model is a dense feed-forward network with RMSE 0.38 on the test data. However, the example XGBoost model does the prediction task with significantly lower RMSE 0.04 on test data.

As described above, in the data, the inventors have seven categorical and three numeric risk factors. Usually, most of the ML and DL algorithms do not accept categorical/factor inputs. Thus, the categorical risk factors are converted to a numerical form. However, in the case, 70% of the risk factors are non-numeric in nature. To overcome this problem, the inventors used a sophisticated technique, termed as “one-hot-encoding.” It is a tool to convert the categorical predictors to numeric in ML algorithms to do a better job in prediction. After the risk factors are converted to numeric scale, Min-Max normalization can be performed on the set of risk factors. Min-Max normalization is a tool used in ML tasks to adjust the predictors and response when they are in different scale. Usually, it makes all the predictors to fall into [0,1]. It is defined as follows:

$$y *= \frac{y - min ( y )}{max ( y ) - min ( y )} \qquad ( 1 - 1 )$$

where y and y* are the original response value, and the normalized value of response respectively. After training the example XGBoost model, the original prediction of the response can be obtained by back transforming. In the data set, the minimum and maximum responses are 0.21 years and 21 years respectively. Hence, min(y)=0.21 years, max(y)=21 years, and max(y)−min(y)=(21−0.21)=20.79 years. Now, back transforming can be performed in the following manner:

y=min(y)+y*[max(y)−min(y)]=0.21+20.79y * (1-2)

In some examples, the z-score standardization can be performed with the data. However, in some scenarios, the min-max normalization can provide better performance with XGBoost. After normalizing the data, the data can be divided into 70% training and 30% test data. At first, the GBM algorithm can be performed on the data. In order to find the example combination of hyperparameters, grid search mechanism can be performed. The grid search mechanism can iterate through every possible combination of hyperparameter values and enable to select the example combination. To perform a grid search, the grid of hyper-parameter combinations can be created. The inventors searched across 3⁵=243 models with varying hyperparameter values. The following Table 1 shows the combinations of the hyperparameters (abbreviated as eta, MD, MCW, SS, and CSBT).

TABLE 1
Hyper-parameters and their combinations in the grid search

| Hyper-parameters | Value Combination |
|---|---|
| eta | (.01, .05, .1) |
| Max Depth (MD) | (3, 5, 7) |
| Min Child Weight (MCW) | (1, 3, 5) |
| Subsample (SS) | (.65, .8, 1) |
| Colsample_bytree (CSBT) | (.65, .8, 1) |

Now, the data analysis with XGBoost can be performed. In some examples, the XGBoost is more sophisticated than the usual GBM and has more options to set the hyper-parameters to reduce overfitting. It has several hyperparameters options to train the model. The brief descriptions of the hyperparameters used for training the model is as follows:
eta: Controls the learning rate, or how quickly the model learns data patterns.
max depth (MW): The depth of the tree is controlled by this variable. Typically, the greater the depth, the more complex the model grows, increasing the likelihood of overfitting.
min child weight (MCW): It denotes the smallest number of instances required in a child node in the context of a regression problem. It aids in preventing overfitting by avoiding potential feature interactions.
subsample (SS): It regulates the number of samples (observations) provided to a tree.
colsample bytree (CSBT): It controls the number of predictors given to a tree.

Now, a grid search mechanism can be performed with different combinations of hyperparameters to find the optimum combination for which the Root Mean Square Error (RMSE) is minimum. The following Table 2 shows top ten models (ascending order of RMSE) with the particular choices of the hyperparameters.

TABLE 2
Top 10 models with hyper-parameters for XGBoost

| eta | M.D | MCW | SS | CSBT | OT | min_RMSE |
|---|---|---|---|---|---|---|
| .05 | 7 | 1 | .8 | .8 | 158 | 0.0304000 |
| .05 | 7 | 3 | 1 | .8 | 182 | 0.0305060 |
| .01 | 7 | 1 | .8 | .65 | 713 | 0.0305134 |
| .05 | 7 | 3 | .8 | .8 | 141 | 0.0306156 |
| .05 | 7 | 3 | 1 | .8 | 134 | 0.0306568 |
| .01 | 7 | 1 | .65 | .65 | 762 | 0.0307100 |
| .01 | 7 | 1 | .8 | .65 | 725 | 0.0307280 |
| .05 | 7 | 1 | .65 | .8 | 174 | 0.0307378 |
| .01 | 7 | 1 | .65 | .8 | 725 | 0.0307526 |
| .01 | 7 | 1 | 1 | .8 | 816 | 0.0307682 |

From Table 2, the minimum RMSE (0.0304) was achieved while training the data when eta=0.05, max depth (MD)=7, min child_weigh (MCW)=1, subsample (SS)=0.8, colsample bytree (CSBT)=0.8, and optimal trees (OT)=158. Therefore, the example XGBoost ensemble model can be expressed as follows:

$${\hat{y}}^{*} = \hat{f} ( x ) = {\sum }_{i = 1}^{158} {\hat{f}}_{i} ( x ) , {\hat{f}}_{i} ∈ ℱ \qquad ( 1 - 3 )$$

where [symbol] is the collection of all possible regression trees and \hat{ƒ}ᵢ are the additive functions (additive trees) in [symbol] . The example analytical model provides the good results with the example values of the six hyper-parameters mentioned above. With the example values of the hyper-parameters, the model can be trained with 5-fold cross-validation, and an RMSE of 0.04127676 can be obtained in test data, which is better than the RMSE using GBM.

An example algorithm is provided to obtain the example analytical model with the example hyper-parameters in the following manner:

Algorithm for obtaining example analytical model

```
Input
  Input Vector: X = (x₁,x₂,...,xₙ).
  response y as output.
  Number of iteration T decided by the researcher.
  Mean Square Error Loss Function L(θ) = Σᵢ₌₁ⁿ(yᵢ − ŷᵢ).
  Decision tree as base (weak) learner to be combined in the ensemble.
Algorithm
  for t = 1 to T do
  1. Initially, a decision tree is fitted to the data: \hat{f}₁ (x) = y.
  2. Next, the subsequent decision tree is fitted to the prior tree's residuals:
d₁(x) = y − \hat{f}₁ (x)
  3. The latest tree is then added to the algorithm: \hat{f}₂ (x) = \hat{f}₁ (x) + d₁(x).
  4. The succeeding decision tree is fitted to the residuals of \hat{f}₂ : d₂(x) = y −
\hat{f}₂(x).
  5. The new tree is then added to our algorithm: \hat{f}₃ (x) = \hat{f}₂ + d₂(x)
  6. Use cross-validation while training the model to decide the stopping
criteria of the training process.
  7. Create a hyper-parameter grid with some user provided values and
perform grid search mechanism to find optimal combination of the hyperparameters.
  8. The final analytical model is the sum of all the decision tree base learners
with optimal values of the hyper-parameter along with optimal number of trees T*: \hat{f} =
Σᵢ₌₁^{T}* \hat{f}ᵢ.
end.
```

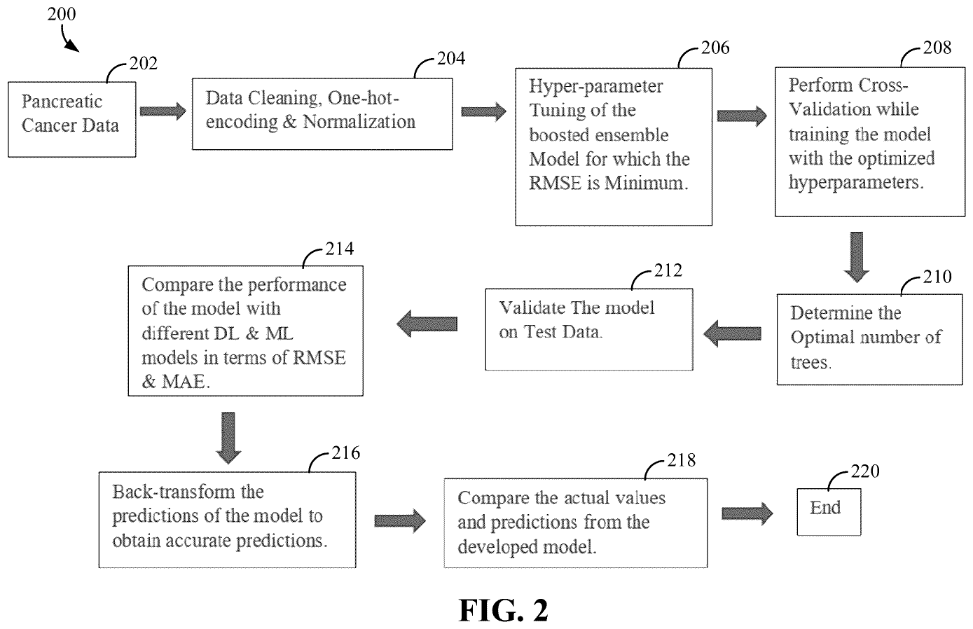

FIG. 2 illustrates an example process 200 for developing the example analytical deep learning model. For example, at step 202, the process 200 can obtain pancreatic cancer data. At step 204, the process 200 can perform data cleaning, one-hot-encoding and normalization. At step 206, the process 200 can perform hyper-parameter tuning of the boosted ensemble model for which the root mean square error (RMSE) is minimum. At step 208, the process 200 can perform cross-validation while training the model with the optimized hyperparameters. At step 210, the process 200 can determine the optimal number of trees. At step 212, the process 200 can validate the model on test data. At step 214, the process 200 can compare the performance of the model with different deep learning and machine learning models in terms of the RMSE and the mean absolute error (MAE). At step 216, the process 200 can back-transform the predictions of the model to obtain accurate predictions. At step 218, the process 200 can compare the actual values and predictions from the developed model. At step 220, the process 200 can end. It should be appreciated that the process 200 can be a mere example. Other suitable additional or alternative steps can be added in the process 200. Also, one or more steps in process 200 can be performed in a different order than presented, in parallel with another step, or bypassed.

Validation of the Example Model: After developing the example analytical model, the model can be validated to implement the model and obtain the best results. In developing the model, 70% of the training data was used and an RMSE of 0.034 was obtained. The example analytical model had a predictive performance in the test data set close to the training data set. For example, when the example model was implemented on the test data set, an RMSE of 0.0422 was obtained. The RMSE of 0.0422 is very close to what the inventors have obtained in the training set, implying that the example model performs well on the unseen/future data set. Based on the example model, the survival times (in years) can be predicted by back-transforming the scaled response using equation (1-2), and how good the prediction is can be compared. The following Table 3 shows the actual and estimated predictions of the pancreatic survival times (in years).

TABLE 3
Predicted and actual responses of the survival times

| Predicted Response | Actual Response |
|---|---|
| 1.58 | 1.78 |
| 2.19 | 2.04 |
| 2.31 | 2.05 |
| 2.57 | 2.16 |
| 2.14 | 2.33 |
| 3.51 | 3.74 |
| 3.21 | 3.39 |
| 2.42 | 2.56 |
| 1.26 | 1.62 |
| 1.55 | 1.85 |
| 2.19 | 2.43 |
| 2.96 | 3.26 |

From the above table, the predictions are very close to the actual response. To validate the prediction accuracy, the inventors also performed Wilcoxon's rank-sum test with continuity correction to check if the actual and predicted responses are significantly different. The test produced a p-value of 0.5 (>0.05), implying that there is insufficient sample evidence to reject the null hypothesis that both actual and predicted responses are the same. Thus, the test suggests there is no significant difference between the actual and predicted responses at a 5% level of significance.

Comparison with different deep learning models: The XGBoost method performed with about 96.42% accurate. The inventors compared the example boosted regression tree (using XGBoost) model with different deep learning models to validate its performance. Deep learning models can be efficient with a large amount of data to train to address the complex structure of features. The inventors used activation functions like rectified linear unit (ReLU), Exponential Linear Unit (ELU), scaled exponential linear units (SELU), and Hyperbolic Tangent (tanh) in different layers of the deep network and used optimizer like stochastic gradient descent (SGD), Root Mean Square Propagation (RMSprop), and Adam (derived from adaptive moment estimation). In some models, the inventors introduced dropouts and batch normalization, and in some models other than the example model. Adding dropouts and batch-normalization can prevent overfitting in the networks and boosts the performance. Each of the models can be trained using 300 epochs and batch size=32. Table 4 compares different deep learning models in terms of root mean square error (RMSE) and mean absolute error (MAE) in the test data. In the following table, the activation function, optimizer, dropout, and batch normalization are abbreviated as AF, OPT., DROP., and BN, respectively. The inventors considered ten deep learning sequential models with three dense layers containing units 100, 90, and 50, respectively. As Table 4 illustrates, the best deep learning model (DL6) with minimum RMSE (0.378) is the model where the inventors use tanh activation function in each of the three hidden layers, use optimizer Adam, use dropout with batch-normalization. The following Table 4 describes different deep-learning models, and compares the RMSE & MAE.

TABLE 4
Comparison of different deep learning models
in terms of RMSE and MAE in the test data

| Model | Unit | AF | OPT. | DROP. | BN | RMSE | MAE |
|---|---|---|---|---|---|---|---|
| DL1 | (100, 90, 50) | (tanh, tanh, relu) | RMSprop | yes | yes | .381 | .26 |
| DL2 | (100, 90, 50) | (ReLU, ReLU, ReLU) | Adam | yes | yes | .391 | .24 |
| DL3 | (100, 90, 50) | (ReLU, ReLU, ReLU) | SGD | yes | yes | .9 | .255 |
| DL4 | (100, 90, 50) | (ReLU, ReLU, ReLU) | RMSprop | Yes | Yes | .391 | .25 |
| DL5 | (100, 90, 50) | (ReLU, ReLU, ReLU) | Adam | No | No | .39 | .26 |
| DL6 | (100, 90, 50) | (tanh, tanh, tanh) | Adam | Yes | Yes | .378 | .249 |
| DL7 | (100, 90, 50) | (ELU, ELU, ReLU) | Adam | Yes | Yes | .388 | .234 |
| DL8 | (100, 90, 50) | (ReLU, SELU, ELU) | Adam | Yes | Yes | .385 | .232 |
| DL9 | (100, 90, 50) | (ReLU, ReLU, ReLU) | Adam | No | Yes | .49 | .4 |
| DL10 | (100, 90, 50) | (ReLU, ReLU, ReLU) | Adam | No | No | .51 | .3 |

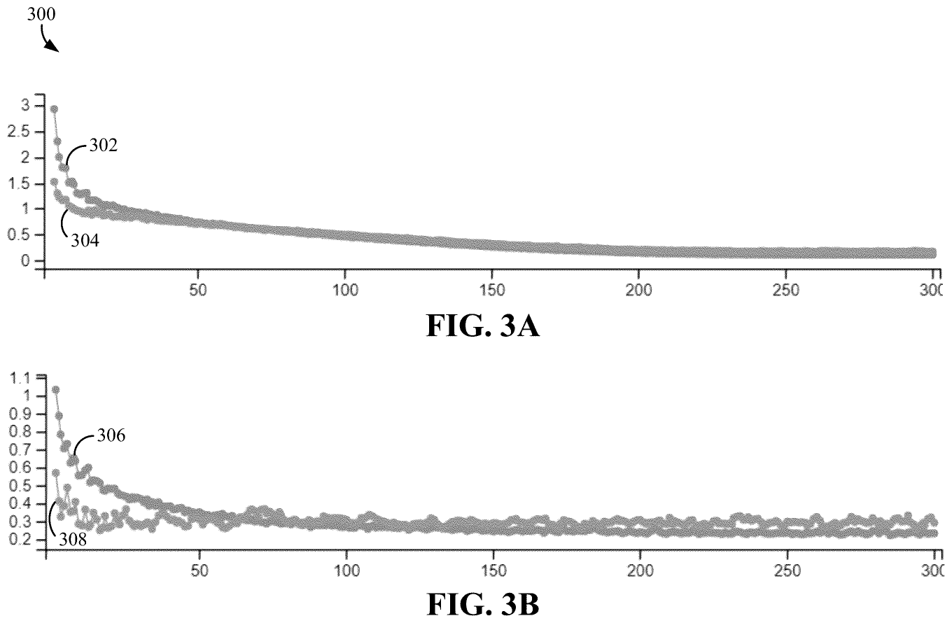

FIGS. 3A and 3B illustrate graphs of RMSE and MAE of the example deep learning model (i.e., DL6 in Table 3) while training the model. For example, FIG. 3A shows a graph with RMSE 302 and validation RMSE 304 of the example deep learning model while FIG. 3B shows a graph with MAE 306 and validation MAE 308 of the example deep learning model.

Ranking of Risk Factors and Prediction of the Survival Time: Once the inventors have found the example model (DL6), the pancreatic risk factors can be ranked according to their relative importance. The contributing risk factors can be ranked in survival time using the measure gain. The gain denotes the relative impact of a certain risk factor to the model, which is computed by considering each predictor's contributions to each tree in the model. A higher value of this metric for a specific risk factor, compared to another risk factor, implies that the risk factor with a higher gain is more important for generating a prediction.

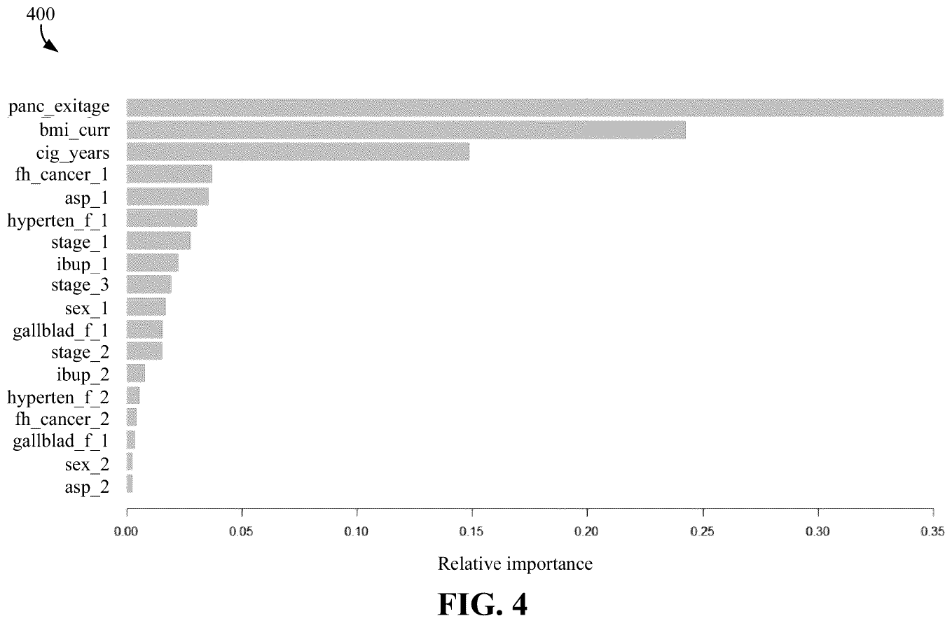

FIG. 4 illustrate the relative importance of risk factors used in the example model (e.g., XGBoost model). From FIG. 4, the top five most contributing risk factors in the model are age, current bmi, the number of years a patient smoked cigarette, people who have family history of cancer, and people who took aspirin on a regular basis. Table 5 illustrates the percentage contributions of the risk factors to the response survival times. From Table 5, the risk factors explain 96.42% of the total variation of the response.

TABLE 5
Risk factors and their percentage of contribution to the response

| Risk Factors | % Contribution |
|---|---|
| panc_exitage | 35.5 |
| bmi curr | 24.3 |
| cig years | 14.93 |
| fh_cancer_1 | 3.76 |
| asp 1 | 3.6 |
| hyperten_f_1 | 3.1 |
| stage_1 | 2.82 |
| Ibup_1 | 2.29 |
| stage_3 | 1.96 |
| sex_1 | 1.73 |
| gallblad_f_1 | 1.6 |
| stage_2 | 1.57 |
| ibup_2 | .83 |
| hyperten_f_2 | .61 |
| fh_cancer_2 | .45 |
| gallblad_f_2 | .4 |
| sex_2 | .29 |
| asp_2 | .28 |

Conclusion: In cancer research, one of the considering aspects is to estimate the survival times of the patients. It results in improved management, more efficient use of resources, and the provision of specialized treatment alternatives. It is desirable to investigate the clinical diagnosis and enhance the therapeutic/treatment strategy of pancreatic cancer. Pancreatic cancer is one of the deadliest forms of cancer, and most of the cases are detected in later stages (stage III/IV). Once a patient is diagnosed with pancreatic cancer, he/she or his/her family members would be interested in knowing how long the expected/predicted survival is. This question is usually asked by patients with a terminal illness to their doctors. However, it is impossible to provide the exact answer to these questions; doctors provide an answer which is mainly subjective. The example model described above is based on real data that answer the questions given a particular choice of risk factors. The example model would be very helpful to the doctors and medical professionals. Also, if some more relevant risk factors are known, those can be incorporated in the example model. This would be helpful for healthcare professionals and patients with terminal illnesses. Given a collection of risk factors, the inventors built a questionnaire that can address the patient information who are diagnosed with pancreatic cancer. Based on their response, the estimate of the survival times can be obtained very accurately. In this disclosure,
1. The inventors have developed a boosted ensemble regression tree model using XGBoost that is very accurate and performs well on test data set, given a collection of risk factors (numeric and categorical).
2. The inventors ranked all the risk factors according to their relative importance in the boosted model. This ranking provides the percentage of contribution of the individual risk factors to the response, survival time.
3. The inventors have compared the performance of the XGBoost model with different machine learning, and other ten deep learning sequential models with different activation functions and optimizers. The XGBoost model produced an RMSE and MAE of 0.0412 and 0.034 which is the smallest on the test data compared to all of the other models.
4. The example analytical model can be implemented to any future data set containing information on different risk factors relating to the subject study to obtain very good predictive performance.

In some examples, the deep learning model can be used for a patient to estimate a survival time of the patient (e.g., using a medical website portal, or any suitable medical system). For example, in some embodiments, the deep learning model may be implemented through a software application running on a processor that implements a user interface. The user interface could prompt a user (e.g., a patient or family member) to input certain health information of a patient. As described herein, the information needed for the model could include basic information that a user could implement, without necessarily requiring a data interface with the user's electronic medical record. Thus, fewer privacy encryptions could be required (Though, in other embodiments, the application could automatically draw this information from an EMR). The user interface could then provide an anticipated survival time for the patient, along with recommended end of life steps and timelines for the patient and family to consider.

In addition, healthcare/pharmaceutical companies, healthcare professionals/doctors, health science researchers, cancer institutes and departments of public health, health policy makers, biomedical research institutes, government health organizations and/or any other suitable organization can use the deep learning model for medical records, commercial products, research, or any other suitable purposes. For example, in one embodiment the deep learning model can be used to compare actual vs predicted outcomes for clinical trials, normalized to the expected outcome of each individual patient. Likewise, risk factors for pancreatic cancer can be used by research agencies to plan preventative actions and monitor survival times as a result. In other embodiments, the deep learning model could run as an integration to an electronic medical record system, to show healthcare providers expected survival times, and make recommendations about whether the survival time warrants attempts at various types of treatments. The survival time prediction could also continually update as patient data changes.

### Example 2: A Stochastic Model for Monitoring the Behavior of Pancreatic Cancer Patients at Different Stages as a Function of Time

In the disclosure, a modern analytical approach using Survival Monitoring Indicator (SMI) is introduced to monitor and evaluate the behavior of survival times pancreatic cancer patients. The inventors considered survival times of patients from three race groups (Caucasian, African-American, and Others) at four different cancer stages, categorized in three different age groups ([40-59), [60-79), and [80-above)). There were 108 patient groups who received three different treatments: only chemotherapy (C), only radiation (R), and a combination of chemotherapy and radiation (C+R). The example analytical method can predict the pattern of survival intensities based on the Survival Monitoring Indicator (SMI) as a function of time t, which can provide information if the specific treatment has been useful for the particular patient group. Also, the concept of Relative Change in Intensities (RCI) is introduced for patients diagnosed with the subject cancer, which gives the approximate change in the stochastic growth intensity function (SGIF) ζ (t), for each unit time change. Finally, the inventors have developed an analytical algorithm to compare the survival intensities for any two specific groups out of a total of 108 patient groups without actually computing the stochastic growth intensities. The example analytical methodology based on Survival Monitoring Indicator (SMI) and stochastic growth intensity function ζ(t) is useful and effective for any subject cancer and can be implemented as a modem approach to monitor and evaluate cancer mortality rate as a function of time. The adaptability of the example technique stems from the fact that the example algorithm may be used to any number of patient groups of any age, of any race, at any specific cancer stage, and receiving any unique treatment or combination.

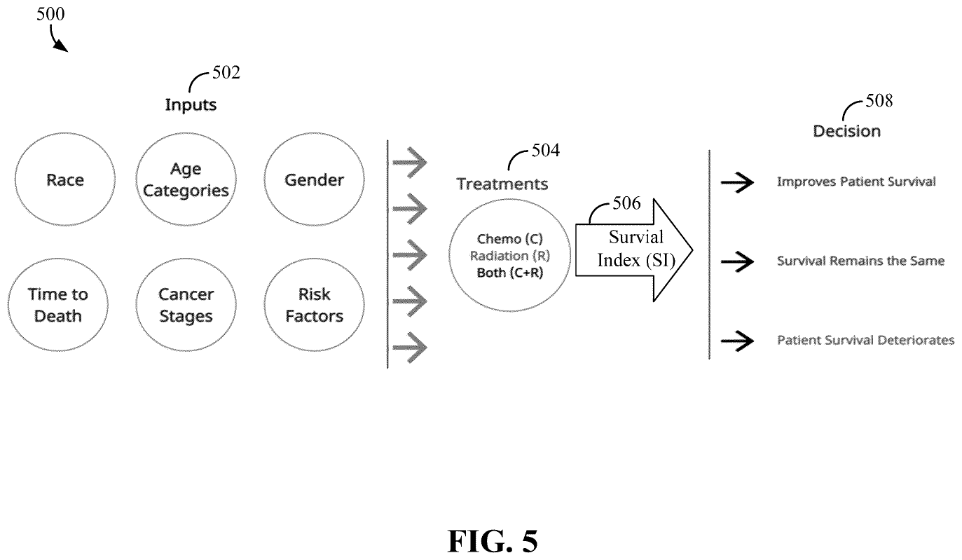

FIG. 5 shows the schematic approach of the example data-driven analytical process to monitor cancer survival time (e.g., pancreatic cancer survival time). For example, one or more inputs 502 (e.g., race, age categories, gender, time to death, cancer stages, risk factors, etc.) for patient groups can be used in the example data-driven analytical process. The patient groups can receive different treatments 504 (e.g., only chemotherapy (C), only radiation (R), and a combination of chemotherapy and radiation (C+R)). The data-driven analytical process can predict a result (e.g., patient survival is improved, patient survival remains the same, or patient survival deteriorates) based on a survival index 506 (e.g., survival monitoring indicator (SMI)) when the patient groups receive the treatments.

Given the destructive nature of cancer (e.g., pancreatic cancer), it remains one of the major threats devastating human existence. However, there are various treatment options (chemotherapy, radiation, surgery, immunotherapy, targeted therapy) to cure the lethal carcinogenic disease. Thus, it is desirable to identify a stage at which a particular treatment option is the most effective. Also, it is desirable to understand how the treatment options are affecting the mortality of patients from a specific race belonging to a particular age group, at different cancer stages, which means by applying a particular treatment of interest or combination of both if the mortality of a patient from a specific race at a specific stage is increasing, decreasing, or staying the same. The present disclosure introduces a new analytical approach by defining the Survival Monitoring Indicator (SMI) to monitor the behavior of the cancer survivorship for patients from different age groups, different cancer stages, and from different races, as a stochastic realization of time.

The inventors used, for an example analytical model, data from the Surveillance, Epidemiology, and End Results (SEER) database, which contains information on patients diagnosed with pancreatic adenocarcinoma. The example analytical model is based on the survival times (in months) and cause-specific death (deaths due to pancreatic cancer) for each patient. The survival times of patients can be one of the considering factors used in all cancer research. It is necessary to evaluate the severity of cancer, which helps to determine the prognosis and help identify the correct treatment options. The inventors have extracted a sufficiently large random sample of patients diagnosed with pancreatic adenocarcinoma from different races (Caucasian, African-American, others), and four cancer stages which contain the information of different treatment options (chemotherapy (C), radiation (R), combination of both (C+R)). The example analytical model categorizes the information for three different age groups: 40 to 59, 60 to 79, and 80 and above. The schematic representations of the data for different races, cancer stages, and age groups are shown in Tables 6 below.

TABLE 6
Showing the number of patients for Caucasian population in
different cancer stages, categorized by age groups
Caucasian

| Age: [40-59) | | | | Age: [60-79) | | | | Age: [80-Above) | | | |
|---|---|---|---|---|---|---|---|---|---|---|---|
| Stages | C | R | C + R | Stages | C | R | C + R | Stages | C | R | C + R |
| I | 148 | 12 | 123 | I | 568 | 75 | 490 | I | 237 | 118 | 162 |
| II | 1206 | 52 | 1351 | II | 3406 | 249 | 3268 | II | 704 | 144 | 478 |
| III | 514 | 33 | 663 | III | 1286 | 117 | 1358 | III | 281 | 451 | 210 |
| IV | 5070 | 123 | 556 | IV | 11263 | 305 | 869 | IV | 1783 | 132 | 120 |

TABLE 7
Showing the number of patients for African-American population
in different cancer stages, categorized by age groups
African American

| Age: [40-59) | | | | Age: [60-79) | | | | Age: [80-Above) | | | |
|---|---|---|---|---|---|---|---|---|---|---|---|
| Stages | C | R | C + R | Stages | C | R | C + R | Stages | C | R | C + R |
| I | 34 | 3 | 29 | I | 88 | 15 | 82 | I | 18 | 18 | 5 |
| II | 211 | 14 | 249 | II | 394 | 45 | 391 | II | 61 | 15 | 33 |
| III | 104 | 13 | 126 | III | 212 | 15 | 203 | III | 17 | 4 | 11 |
| IV | 1000 | 45 | 101 | IV | 1476 | 68 | 113 | IV | 157 | 15 | 10 |

TABLE 8
Showing the number of patients for other (American Indian/AK
Native, Asian/Pacific Islander) race groups in different
cancer stages, categorized by age groups
Others

| Age: [40-59) | | | | Age: [60-79) | | | | Age: [80-Above) | | | |
|---|---|---|---|---|---|---|---|---|---|---|---|
| Stages | C | R | C + R | Stages | C | R | C + R | Stages | C | R | C + R |
| I | 16 | 1 | 12 | I | 50 | 9 | 37 | I | 25 | 14 | 6 |
| II | 118 | 10 | 104 | II | 263 | 22 | 244 | II | 63 | 16 | 43 |
| III | 44 | 6 | 65 | III | 149 | 18 | 147 | III | 27 | 6 | 21 |
| IV | 461 | 16 | 62 | IV | 918 | 34 | 101 | IV | 134 | 16 | 20 |

Analytical Method for Developing the Survival Monitoring Indicator (SMI): In the context of pancreatic cancer research, research scientists would like to investigate the survival rate pattern as a function of time for patients belonging to a specific race, age group, cancer stages, and specific treatments they received. For example, researchers would be interested in monitoring and evaluating if the failure rate of survival time of a patient belonging to the Caucasian race receiving chemotherapy from age group [60-79) at Stage IV shows an increasing or decreasing trend. As a result, it is desirable to track how the survival rate changes over time as a result of the application of a certain treatment. In this regard, stochastic growth intensity factor (SGIF) can be defined such that SGIF measures the rate of change of a survival time as a stochastic realization of time. The analytical structure of the (SGIF) function is:

$$ζ ( t ; 𝒮ℳℐ ; ϕ ) = \frac{𝒮ℳℐ}{ϑ} {( \frac{t}{ϕ} )}^{𝒮ℳℐ - 1} , \qquad ( 2 - 1 )$$

$$𝒮ℳℐ > 0 , ϑ > 0 , t > 0$$

where SMI and ϕ are the shape and scale parameters, respectively, and t denotes the time behavior of the incident under investigation.

For n survival times, t₁<t₂< . . . <tₙ, (where t₁<t₂< . . . <tₙ to are the observed and successive), the joint probability density function, ƒ(t₁, . . . , tₙ) can be expressed in terms of ζ(t; SMI; ϕ) as follows,

$$( 2 - 2 )$$

$$\begin{matrix}f ( {t}_{1} , … , {t}_{n} ) = {\prod }_{i = 1}^{n} ( ζ ( {t}_{i} ) exp [ - {\int }_{0}^{{t}_{n}} ζ ( y ) dy ] \\ = {\prod }_{i = 1}^{n} \frac{𝒮ℳℐ}{ϕ} {( \frac{{t}_{i}}{ϕ} )}^{𝒮ℳℐ - 1} exp [ - {\int }_{0}^{{t}_{n}} \frac{𝒜ℐ}{ϕ} {( \frac{y}{ϕ} )}^{𝒮ℳℐ - 1} dy ] \\ = \frac{{𝒮ℳℐ}^{n}}{{ϕ}^{n 𝒮ℳℐ}} {( {\prod }_{i = 1}^{n} )}^{𝒮ℳℐ - 1} exp [ - {( \frac{{t}_{n}}{ϕ} )}^{𝒮ℳℐ} ] ,\end{matrix}$$

$$where {t}_{1} < {t}_{2} < … < {t}_{n} .$$

Implementing the method of Maximum Likelihood Method (MLE) of parameter estimation, the parameters SMI and q can be estimated. The likelihood function when T₁=t₁; T₂=t₂, . . . , Tₙ=tₙ can be expressed as:

$$\begin{matrix}ℒ = L ( t ; 𝒮ℳℐ ; ϕ ) = {\prod }_{i = 1}^{n} {f}_{i} ( t \vert {t}_{1} , … , {t}_{i - 1} ) \\ = {( \frac{𝒮ℳℐ}{ϕ} )}^{n} {\prod }_{i = 1}^{n} {( \frac{{t}_{i}}{ϕ} )}^{𝒮ℳℐ - 1} exp [ - {( \frac{{t}_{n}}{ϕ} )}^{𝒮ℳℐ} ]\end{matrix} \qquad ( 2 - 3 )$$

The parameter, SMI is a function of tₙ, the maximum failure time or the largest value of the phenomenon of interest. We compute the estimate of SMI by equating the partial derivative of L with respect to SMI and setting it equal to zero, then solving for SMI, given by,

$$\frac{\partial ℒ}{\partial ℐ} = 0 ; 𝒮 \hat{ℳ} ℐ = \frac{n}{{\sum }_{i = 1}^{n} log ( \frac{{t}_{n}}{{t}_{i}} )} \qquad ( 2 - 4 )$$

The parameter ϕ is a function of SMI. In a similar way, as above, the estimate of ϕ is computed by equating the partial derivative of L with respect to ϕ0 to zero and then, substituting the estimate of SMI, given by,

$$\frac{\partial ℒ}{\partial ϕ} = 0 ; \hat{ϕ} = \frac{{t}_{n}}{{n}^{\frac{1}{x}}} \qquad ( 2 - 5 )$$

In the context of cancer survivorship, we formally define the Survival Monitoring Indicator (SMI) as follows.

Definition 1 The Survival Monitoring Indicator (SMI) for a patient group belonging to a particular race, from a specific age group is an index based on the survival time, that determines the improvement or deterioration of survival of that particular group at a specified cancer stage when any definite treatment or a combination of more than one treatment is administered.

Mathematically, it can be expressed as follows.

$${𝒮ℳℐ}_{jk}^{lm} = \frac{{n}_{jk}^{lm}}{{\sum }_{i = 1}^{n} log ( \frac{{( {t}_{n} )}_{jk}^{lm}}{{( {t}_{i} )}_{jk}^{lm}} )} \qquad ( 2 - 6 )$$

where SMIⱼₖ^{lm} is the SMI of the j^{th}, (j=1=C, 2=R, 3=C+R) treatment group, at Stage k, k=1, 2, 3, 4, for age group l, (l=1=[40-59]), 2=[60-79], 3=[80-above]) belonging to race m, (m=1=white, 2=black, and 3=other). The term (tₙ)ⱼₖ^{lm} is the largest time to death, and mⱼₖ^{lm} is the number of patients. For example, SMI₃₂¹² is represents the index indicator value for the black patient group, under age group [80-above) at Stage II who received only chemotherapy. Now, we can express the stochastic growth intensity function (SGIF) for any specific group in the following way:

$$ζ ( t ; {𝒮ℳℐ}_{jk}^{lm} ; ϕ ) = \frac{{𝒮ℳℐ}_{jk}^{lm}}{ϕ} {( \frac{t}{ϕ} )}^{{𝒮ℳℐ}_{jk}^{lm} - 1} , \qquad ( 2 - 7 )$$

$${𝒮ℳℐ}_{jk}^{lm} > 0 , ϕ > 0 , t > 0$$

In some examples, SMIⱼₖ^{lm} depends on the interpretation of the SGIF ζ.

Case 1: ζ(t) is decreasing with time, that is, the patient survival rate is improving as a function of time t.

ζ(t) being a decreasing function of t can be expressed as:

$$ζ ( t ) < ζ ( t - 1 ) , \text{} for t - 1 < t ⇒ \frac{{𝒮ℳℐ}_{jk}^{lm}}{ϕ} {( \frac{t}{ϕ} )}^{{𝒮ℳℐ}_{jk}^{lm} - 1} < \frac{{𝒮ℳℐ}_{jk}^{lm}}{ϕ} {( \frac{t - 1}{ϕ} )}^{{𝒮ℳℐ}_{jk}^{lm} - 1} ⇒ {( \frac{t}{ϕ} )}^{{𝒮ℳℐ}_{jk}^{lm} - 1} < {( \frac{t - 1}{ϕ} )}^{{𝒮ℳℐ}_{jk}^{lm} - 1} ⇒ {( \frac{t - 1}{t} )}^{{𝒮ℳℐ}_{jk}^{lm} - 1} > 0$$

Replacing t with (t−1), in the above inequality, can provide

$${( \frac{t - 2}{t - 1} )}^{{SMI}_{jk}^{lm} - 1} > 0.$$

Again, replacing (t−1) with (t−2), in above inequality can provide

$${( \frac{t - 3}{t - 2} )}^{{SMI}_{jk}^{lm} - 1} > 0.$$

Proceeding in a similar manner can provide

$${( \frac{{t}_{1}}{{t}_{0}} )}^{{SMI}_{jk}^{lm} - 1} > 0 ,$$

where t₀ is the initial time of death

Arranging all the above inequalities and expressing them in product form can provide,

$$[ {( \frac{t - 1}{t} )}^{{𝒮ℳℐ}_{jk}^{lm} - 1} {( \frac{t - 2}{t - 1} )}^{{𝒮ℳℐ}_{jk}^{lm} - 1} … {( \frac{{t}_{2}}{{t}_{1}} )}^{{𝒮ℳℐ}_{jk}^{lm} - 1} {( \frac{{t}_{1}}{{t}_{0}} )}^{{𝒮ℳℐ}_{jk}^{lm} - 1} ] > 0 ⇒ {( \frac{1}{{tt}_{0}} )}^{{𝒮ℳℐ}_{jk}^{lm} - 1} > 0$$

Since t₀>0, in order to satisfy the above inequality, SMIⱼₖ^{lm} can satisfy, SMIⱼₖ^{lm}−1<0→SMIⱼₖ^{lm}<1.

Case 2: ζ(t) is increasing with time, that is, the patient survival rate is deteriorating as a function of time t.

For V (t) being an increasing function of t, proceeding with the similar logic can provide SMIⱼₖ^{lm}>1.

Case 3: ζ(t) is constant; that is, the patient survival rate is constant.

For ζ(t) being an independent function of t, proceeding with a similar argument, we end up having SMIⱼₖ^{lm}≈1. Now, provided the estimates of SMIⱼₖ^{lm} and ϕ, the value of the SGIF, ζ(·) (given in equation (2-7)) can be calculated. ζ(·) can be utilized in modeling the survival growth of a specific patient group, receiving any treatment or combination at any given time t. ζ(t) can be a measure of the rate of change in survival growth as a function of time when a patient deteriorates/improves with the use of any given treatment (radiation/chemotherapy/combination of both). A decrease in ζ(t) can imply that SGIF is decreasing or an improvement in the survival rate of a patient diagnosed with pancreatic cancer as a function of time. This means that SMI<1. A rise in ζ(t) can suggest that SGIF is increasing, implying that SMI>1. This means that the survival rate is decreasing with respect to time. When there is no change in ζ(t), it can imply that SMI≈1; thus SGIF is constant. Therefore, the behavior of the change in the cancer survival growth model can be dependent on SMI of the SGIF. That is, SMI can be used to monitor the survival rate of patients as a function of time.

Analytical Method for Developing the Relative Change in Intensity (RCI): The SGIF ζ(t) be used in deciding the pattern of the mortality rate of a group of patients as a function of time under the application of a specific treatment group. Depending upon the values of the Survival Monitoring Indicator (SMI) ( [symbol] 1), it can predict that if the survival rate increases, decreases, or staying constant. However, what can be said about the SGIFs of two groups of patients receiving two different treatments where both the Survival Monitoring Indicator (SMI) is less than 1, or greater than 1? In the present disclosure, how the SGIF changes for two different (SMI) is illustrated, where both (SMI) is ≤1 or >1.

For any two different groups (can be of a different race, age-group, cancer stage, or treatment group) let SMI₁, and SMI₂ (for calculation simplicity, we use only one suffix, instead of four) be the survival monitoring indicator for two different groups, and ϕ₁ and ϕ₂ be the corresponding scale parameters. The corresponding SGIFs be δ₁(t), and ζ₂(t), respectively.

Case 1: SMI₁<SMI₂

From Equation (2-7), Equation (2-8) can be obtained:

$$\begin{matrix}\frac{{ζ}_{1} ( t )}{{ζ}_{2} ( t )} = {\underbrace{\begin{matrix}( \frac{{𝒮ℳℐ}_{1}}{{𝒮ℳℐ}_{2}} ) & ( \frac{{θ}_{2}^{{𝒮ℳℐ}_{2}}}{{θ}_{1}^{{𝒮ℳℐ}_{1}}} )\end{matrix}}}_{Constant} {t}^{( {𝒮ℳℐ}_{1} - {𝒮ℳℐ}_{2} )} \\ = {Ct}^{( {𝒮ℳℐ}_{1} - {𝒮ℳℐ}_{2} )} .\end{matrix} \qquad ( 2 - 8 )$$

From (2-8),

$$\frac{{ζ}_{1} ( t )}{{ζ}_{2} ( t )}$$

is a decreasing function of time, since

$${SMI}_{1} < {SMI}_{2} . Let h ( t ) = \frac{{ζ}_{1} ( t )}{{ζ}_{2} ( t )} .$$

Then, Equation (2-9) can be obtained:

$${h}^{′} ( t ) = \frac{{ζ}_{2} ( t ) {{ζ}_{1} ( t )}^{′} - {ζ}_{1} ( t ) {{ζ}_{2} ( t )}^{′}}{{( {ζ}_{2} ( t ) )}^{2}} < 0 ⟹ {ζ}_{2} ( t ) {{ζ}_{1} ( t )}^{′} < {ζ}_{1} ( t ) {{ζ}_{2} ( t )}^{′} ⟹ \frac{{{ζ}_{1} ( t )}^{′}}{{ζ}_{1} ( t )} < \frac{{{ζ}_{2} ( t )}^{′}}{{ζ}_{2} ( t )} ⟹ t \frac{{{ζ}_{1} ( t )}^{′}}{{ζ}_{1} ( t )} < t \frac{{{ζ}_{2} ( t )}^{′}}{{ζ}_{2} ( t )} . \qquad ( 2 - 9 )$$

The term

$$t \frac{ζ ( t ) ′}{ζ ( t )}$$

in the above expression can be expressed as:

$$\begin{matrix}t \frac{{ζ ( t )}^{′}}{ζ ( t )} = {lim}_{x \to t} [ \frac{ζ ( x ) - ζ ( t )}{x - t} \frac{t}{ζ ( t )} ] \\ = {lim}_{x \to t} [ \frac{ζ ( x ) - ζ ( t )}{ζ ( t )} \frac{t}{x - t} ] \\ = {lim}_{x \to t} \frac{1 - \frac{ζ ( x )}{V ( t )}}{1 - \frac{x}{t}} \\ ≅ \frac{\% Δ ζ ( t )}{\% Δ t}\end{matrix} .$$

In the above expression

$$\frac{\% Δ ζ ( t )}{\% Δ t}$$

is the ratio of relative percent change in ζ(t) with respect to relative percent change in t. It can also be thought of as the approximate change in the SGIF ζ(t) for each unit time change. The term can be as Relative Change in Intensity (RCI). Thus, RCI can be expressed as:

$$RCI = t \frac{{ζ ( t )}^{′}}{ζ ( t )} \qquad ( 2 - 10 )$$

From (2-10), it can be noted that,

$$\begin{matrix}RCI = t \frac{{ζ ( t )}^{′}}{ζ ( t )} \\ = [ \frac{\frac{{ζ ( t )}^{′}}{ζ ( t )}}{\frac{1}{t}} ] \\ = \frac{\frac{d}{dt} log ζ ( t )}{\frac{d}{dt} log t}\end{matrix} \qquad ( 2 - 11 )$$

In some examples, RCI can be seen as the rate of change of the intensity function ζ(t) in the logarithmic scale with respect to the rate of the chance of time in the logarithmic scale. Now, the Relative Change in Intensity (RCI) can be defined formally.

Definition 2 RCI is the ratio of the relative percent change in the death rate ζ(t) with respect to relative percent change in the survival time t.

Definition 3 RCI can also be defined as the ratio of the SGIF ζ(t) and the rate of the survival time in logarithmic scale.

In this context, Equation (2-12) can be expressed as:

$$\frac{{ζ ( t )}^{′}}{ζ ( t )} = \frac{d}{dt} ln ζ ( t ) \qquad ( 2 - 12 )$$

Equation (2-12) is the exact rate of change of the log of the intensity function ζ(t) with respect to time t. From Equation (2-11), RCI₁<RCI₂ can be obtained when 0<SMI₁<SMI₂. That is, if prior knowledge about the survival monitoring indicator SMI for any two different patient groups exists where one is less than the other, the relative change in intensity (RCI) for the patient group, for which SMI is less, can be smaller than the competitive group.

Case 2: SMI₁>SMI₂:

Following a similar approach as in Case 1, RCI₁>RCI₂ can be obtained. That is, the relative change in intensity (RCI) for the patient group which has greater SMI index, can be greater than the competitive group.

Case 3: SMI₁=SMI₂:

Following the similar approach as in Case 1, RCI₁=RCI₂ can be obtained. That is, the relative change in intensity (RCI) for two patient groups can be the same if they have the same Survival Monitoring Indicator SMI.

Deriving the Criterion for the Stochastic Growth Intensity ζ(t) and Time t Based on the Survival Monitoring Indicator (SMI): Now, the criterion can be derived on relative change in intensity (RCI). The range of time t can be determined under Case 1; that is, when SMI₁<SMI₂ assuming ζ₁(t)≤ζ₂(t) and ζ₁(t)≤ζ₂(t). Equation (2-13) can be expressed as:

$$\frac{{ϕ}_{2}^{{𝒮ℳℐ}_{2}}}{{ϕ}_{1}^{{𝒮ℳℐ}_{1}}} = \frac{{t}_{n 2}}{{( {n}_{2} )}^{1 / ϕ}} \frac{{( {n}_{1} )}^{1 / {ϕ}_{2}}}{{t}_{{n}_{1}}} \qquad ( 2 - 13 )$$

Combining Equation (2-10) and (2-13), Equation (2-14) can be expressed as:

$$\begin{matrix}\frac{{ζ}_{1} ( t )}{{ζ}_{2} ( t )} = ( \frac{{𝒮ℳℐ}_{1}}{{𝒮ℳℐ}_{2}} ) \frac{{t}_{{n}_{2}}}{{( {n}_{2} )}^{1 / {ϕ}_{2}}} \frac{{( {n}_{1} )}^{1 / {ϕ}_{1}}}{{t}_{{n}_{1}}} {t}^{( {𝒮ℳℐ}_{1} - {𝒮ℳℐ}_{2} )} \\ = δ {t}^{( {𝒮ℳℐ}_{1} - {𝒮ℳℐ}_{2} )}\end{matrix} , \qquad ( 2 - 14 )$$

$$where$$

$$δ = \frac{{𝒮ℳℐ}_{1}}{{𝒮ℳℐ}_{2}} \frac{{t}_{{n}_{2}}}{{( {n}_{2} )}^{1 / {𝒮ℳℐ}_{2}}} \frac{{( {n}_{1} )}^{1 / {𝒮ℳℐ}_{2}}}{{t}_{{n}_{1}}}$$

can be a constant quantity. In some examples, the range can be found if t under the following equation or assumption:

ζ₁(t)≤ζ₂(t), and SMI₁<SMI₂

By writing the above expression, while comparing any two patient groups, the group that has the lesser SMI value, can have also the lesser SGIF ζ(t). The following can be considered.

$${ζ}_{1} ( t ) \leq {ζ}_{2} ( t ) \qquad ( 2 - 15 )$$

$$⇔ \frac{{ζ}_{1} ( t )}{{ζ}_{2} ( t )} \leq 1$$

$$⇔ δ {t}^{( {𝒮ℳℐ}_{1} - {𝒮ℳℐ}_{2} )} \leq 1 , ( from ( 16 ) )$$

$$⇔ {t}^{( {𝒮ℳℐ}_{1} - {𝒮ℳℐ}_{2} )} \leq \frac{1}{δ} ( as δ > 0 )$$

$$⇔ ( {𝒮ℳℐ}_{1} - {𝒮ℳℐ}_{2} ) log t \leq log ( \frac{1}{δ} )$$

$$\begin{matrix}⇔ log t \geq \frac{log ( \frac{1}{δ} )}{( {𝒮ℳℐ}_{1} - {𝒮ℳℐ}_{2} )} \\ = \frac{- log δ}{( {𝒮ℳℐ}_{1} - {𝒮ℳℐ}_{2} )} \\ = \begin{matrix}\frac{log δ}{( {𝒮ℳℐ}_{2} - {𝒮ℳℐ}_{1} )} , & as ( {𝒮ℳℐ}_{1} - {𝒮ℳℐ}_{2} ) < 0\end{matrix}\end{matrix}$$

$$⇔ t \geq {e}^{\frac{log δ}{( {𝒮ℳℐ}_{2} - {𝒮ℳℐ}_{1} )}} = {( {e}^{log δ} )}^{\frac{1}{( {𝒮ℳℐ}_{2} - {𝒮ℳℐ}_{1} )}} = {δ}^{\frac{1}{( {𝒮ℳℐ}_{2} - {𝒮ℳℐ}_{1} )}}$$

From (2-15),

$${ζ}_{1} ( t ) \leq {ζ}_{2} ( t ) ⇔ t ∈ [ {δ}^{\frac{1}{( {𝒮ℳℐ}_{2} - {𝒮ℳℐ}_{1} )}} , ∞ )$$

Now, the case when ζ₁(t)≥ζ₂(t) can be considered. Then, proceeding in a similar manner as the previous case, the following condition can be obtained:

$${ζ}_{1} ( t ) \geq {ζ}_{2} ( t ) ⇔ t ∈ ( 0 , {δ}^{\frac{1}{( {𝒮ℳℐ}_{2} - {𝒮ℳℐ}_{1} )}} ] .$$

The above conditions can be the necessary and sufficient conditions for comparing any two intensities ζ₁(t) and ζ₂(t) and obtaining the range of the survival time t. That is, if the prior information exists about the range of the survival time t of any two specific patient groups, their death intensities ζ₁(t) and ζ₂(t) at time t can be compared. Conversely, if the knowledge that the SGIF ζ(t) of any specific patient group is less/more than the other exists, the range of the survival time (time to death) for both of the patient groups can be found. This approach can be extended to more than one patient groups for comparison purpose.

Results: The following tables (Tables 9-11) show the SMI values for a Caucasian race group at the four cancer Stages, categorized by three age groups ([40-59), [60-79), and [80-above)), who receive three treatment options (only chemotherapy (C), only radiation (R), and the combination of both (C+R)).

TABLE 9
Showing the SMI and ϕ Values for the Caucasian Race Groups
at Different Cancer Stages, for Age Group [40-59)
WHITE
Age: [40-59)

| | Treatment | | | | | |
|---|---|---|---|---|---|---|
| | C | | R | | C + R | |
| Parameter | SMI | ϕ | SMI | ϕ | SMI | ϕ |
| Stage I | .54 | .007 | 1.09 | 1.94 | .47 | .004 |
| Stage II | .45 | 1.5 × 10⁻⁵ | .41 | .008 | .36 | 2.4 × 10⁻⁷ |
| Stage III | .39 | 1.4 × 10⁻⁵ | .48 | .03 | .42 | .001 |
| Stage IV | .33 | 7.2 × 10⁻¹⁰ | .29 | 5.28 × 10⁻⁶ | .37 | 4.19 × 10⁻⁶ |

TABLE 10
Showing the SMI and ϕ Values for the Caucasian Race Groups
at Different Cancer Stages, for Age Group [50-79)
WHITE
Age: [60-79)

| | Treatment | | | | | |
|---|---|---|---|---|---|---|
| | C | | R | | C + R | |
| Parameter | SMI | ϕ | SMI | ϕ | SMI | ϕ |
| Stage I | .4 | 1.74 × 10⁻⁵ | .52 | .01 | .41 | 4.2 × 10⁻⁵ |
| Stage II | .4 | 2.15 × 10⁻⁷ | .37 | 4.2 × 10⁻⁵ | .46 | 2.8 × 10⁻⁶ |
| Stage III | .37 | 4.8 × 10⁻⁷ | .33 | 7.8 × 10⁻⁵ | .42 | 2.8 × 10⁻⁶ |
| Stage IV | .3 | 4.3 × 10⁻¹² | .32 | 9.6 × 10⁻⁷ | .43 | 9.65 × 10⁻⁶ |

TABLE 11
Showing the SMI and ϕ Values for the Caucasian Race Groups
at Different Cancer Stages, for Age Group [80-above)
Caucasian
Age: [80-Above)

| | Treatment | | | | | |
|---|---|---|---|---|---|---|
| | C | | R | | C + R | |
| Parameter | SMI | ϕ | SMI | ϕ | SMI | ϕ |
| Stage I | .37 | 4.39 × 10⁻⁵ | .41 | .0008 | .4 | .0004 |
| Stage II | .4 | 8.2 × 10⁻⁶ | .43 | .0007 | .46 | .0001 |
| Stage III | .4 | 6.4 × 10⁻⁴ | .5 | .02 | .53 | .002 |
| Stage IV | .33 | 2.2 × 10⁻⁸ | .32 | 1.5 × 10⁻⁵ | .4 | .0004 |

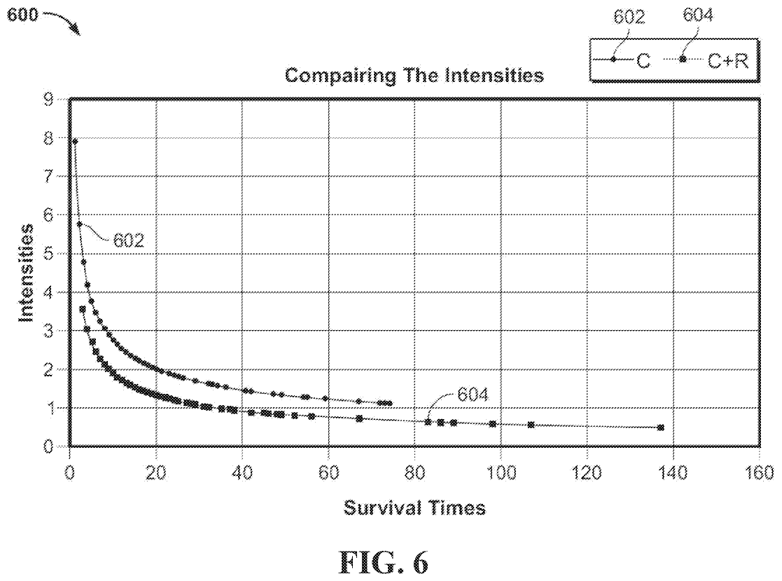

From Table 9, at Stage I, for age group [40-59), the SMI is 0.47 for the patient who received chemotherapy and radiation (C+R) together, which is less than the SMI (0.54) of the patient group who received only chemotherapy (C). As a consequence, the Relative Change in Intensity (RCI) for C+R group is less than the only C group, which follows from Equation (2-11). In other words, the approximate change in the failure intensity V (t), for each unit time change for group C+R at Stage I, for age group [40-59) can be less than the group who receive only C for Caucasian race, which implies that chemotherapy together with radiation has been more effective at Stage I for the particular age group which is also evident from FIG. 6. FIG. 6 is a graph showing the SGIFs for Caucasian Race at Stage I, Under Age Group [4059), who received Only Chemotherapy, and the group who received Chemotherapy & Radiation. In some examples, the SMI=1.09 (>1) for only radiation (R) group at Stage I implying that the survival intensity is decreasing with time for the particular age group receiving radiation therapy only which is not effective with respect to the survival. In the example analytical method, it can be implemented for any chosen group at any given cancer stage, from any particular age group receiving any specific treatment. The following tables (Tables 12-14) shows the SMI values for African-American race group at the four cancer Stages, categorized by three age groups ([40-59), [60-79), and [80-above)), who receive three treatment options (only chemotherapy (C), only radiation (R), and the combination of both (C+R)).

TABLE 12
Showing the SMI and ϕ Values for the African-American Race Groups
in Different Cancer Stages, for Age Group [40-59)
African-American
Age: [40-59)

| | Treatment | | | | | |
|---|---|---|---|---|---|---|
| | C | | R | | C + R | |
| Parameter | SMI | ϕ | SMI | ϕ | SMI | ϕ |
| Stage I | .12 | .6 | 4.52 | 12.01 | .56 | .19 |
| Stage II | .54 | .004 | .91 | 1.15 | .48 | .001 |
| Stage III | .4 | .001 | .83 | 1.18 | .5 | .005 |
| Stage IV | .36 | 4.45 × 10⁻⁷ | .41 | .005 | .38 | .0004 |

TABLE 13
Showing the SMI and ϕ Values for the African-American Race Groups
in Different Cancer Stages, for Age Groups [60-79)
African-American
Age: [60-79)

| | Treatment | | | | | |
|---|---|---|---|---|---|---|
| | C | | R | | C + R | |
| Parameter | SMI | ϕ | SMI | ϕ | SMI | ϕ |
| Stage I | .4 | .0016 | 1.08 | 1.19 | .43 | .005 |
| Stage II | .42 | .00005 | .38 | .0046 | .53 | .001 |
| Stage III | .42 | .00024 | 1.19 | 1.03 | .65 | .014 |
| Stage IV | .35 | 7.67 × 10⁻⁸ | .32 | .0001 | .64 | .02 |

TABLE 14
Showing the SMI and ϕ Values for the African-American Race Groups
in Different Cancer Stages, for Age Groups [80-above)
African-American
Age: [80-Above)

| | Treatment | | | | | |
|---|---|---|---|---|---|---|
| | C | | R | | C + R | |
| Parameter | SMI | ϕ | SMI | ϕ | SMI | ϕ |
| Stage I | .78 | .69 | .65 | .36 | 1.21 | 8.19 |
| Stage II | .65 | .06 | .73 | .61 | .48 | .05 |
| Stage III | 1.31 | 1.95 | 1.39 | 6.31 | .74 | 1.49 |
| Stage IV | .43 | .0005 | .81 | .35 | 1.2 | 2.05 |

From the above Table 12, the survival monitoring indicator (SMI) is greater than 1 for African-American patients from age group [40-59), at Stage I receiving only radiation therapy (SMI=4.52), patients from age group [60-79), at Stage I receiving only radiation therapy (SMI=1.08) (Table 14), patients from age group [60-79), at Stage III receiving only radiation therapy (SMI=1.19), patients from age group [80-above) (Table 15), at Stage I receiving both chemotherapy and radiation (SMI=1.21), patients from age group [80-above), at Stage III receiving only chemotherapy (SMI=1.31) and, only radiation therapy (SMI=1.39). At Stage IV, under the age group [80-above), patients who received both chemotherapy and radiation (C+R), the SMI is 1.2. These results can produce an indication regarding implementing the specific treatments to the specific patient groups of African-American race for which SMI is more than 1, implying that the survival rate is deteriorating for these patients.

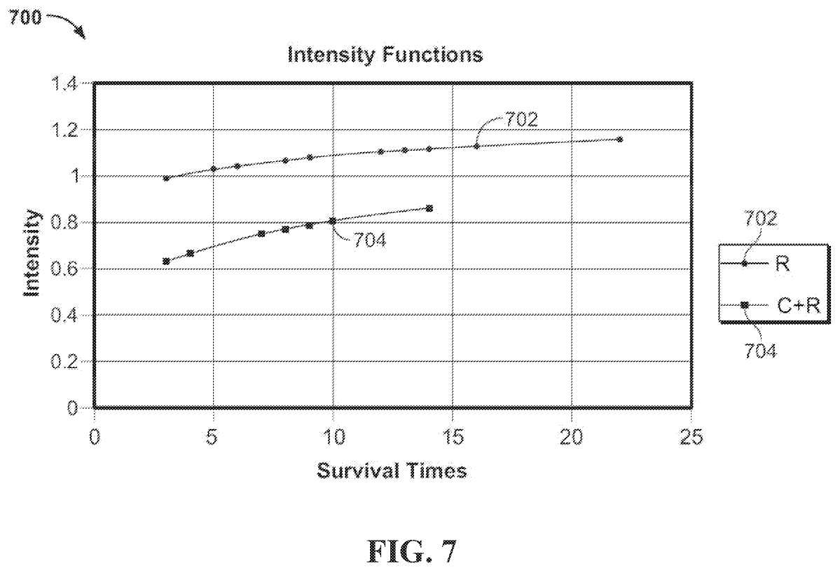

In some examples, the intensities of two specific groups of African-American patients can be compared as shown in FIG. 7. FIG. 7 shows the Comparison between the SGIFs for African-American Race at Stage I, Under Age Group [60-79), Who Received Only Radiation, and the group who received Chemotherapy & Radiation at Stage IV, under age group [80-above). From FIG. 7, the failure intensity curve of the patients who received chemotherapy and radiation (C+R) together at Stage IV, under age group [80-above) lies below than the patients who received only radiation (R) at Stage I, under age group [60-79). However, the SMI (1.2) for C+R group is greater than that of SMI (1.08) for R group, the intensity graph for C+R group lies below the graph of R; which can mean SMI₁<SMI₂ does not imply ζ₁(t)<ζ₂(t) as ζ(t; SMI, ϕ) depends on the time t and another parameter ϕ. However, as Equation (2-11) suggests, RCI₁<RCI₂ if SMI₁<SMI₂. In some examples, SMI₁=1.08<SMI₂=1.2. When RCI₁ and RCI₂ are for time t=3, an equation can be expressed as:

$$RCI = \frac{{ζ}^{′} ( t )}{ζ ( t )} = t ( 𝒮ℳℐ - 1 ) {t}^{- 1}$$

From the equation, RCI₁(t)=RCI₁(3)=3×(1.08−1)/3=0.078 and RCI₂(3)=3×(1.2−1)/3=0.201>RCI₁(3). Hence the relative percentage change in the failure intensity for the patients who received only radiation at Stage I with respect to the relative percent change in t=3 months is approximately 7.8%. On the other hand, the relative percentage change in the failure intensity for the patients who received both C+R at Stage IV with respect to the relative percent change in t=3 months approximately 20%.

The following tables (Tables 15-17) shows the SMI values for Other (American Indian/AK Native, Asian/Pacific Islander) race group at the four cancer Stages, categorized by three age groups ([40-59), [60-79), and [80-above)), who receive three treatment options (only chemotherapy (C), only radiation (R), and the combination of both (C+R)).

TABLE 15
Showing the SMI and ϕ Values for Other (American Indian/AK
Native, Asian/Pacific Islander) Race Groups in Different
Cancer Stages, for Age Group [4059)
OTHERS
Age: [40-59)

| | Treatment | | | | | |
|---|---|---|---|---|---|---|
| | C | | R | | C + R | |
| Parameter | SMI | ϕ | SMI | ϕ | SMI | ϕ |
| Stage I | .55 | .74 | — | — | .5 | .98 |
| Stage II | .59 | .02 | .96 | 2.6 | .66 | .07 |
| Stage III | .63 | .14 | 1.44 | 5.76 | .7 | .12 |
| Stage IV | .35 | 2.62 × 10⁻⁶ | .33 | .02 | .54 | .02 |

TABLE 16
Showing the SMI and ϕ Values for Other (American Indian/AK
Native, Asian/Pacific Islander) Race Groups in Different
Cancer Stages for Age Group [6079)
OTHERS
Age: [60-79)

| | Treatment | | | | | |
|---|---|---|---|---|---|---|
| | C | | R | | C + R | |
| Parameter | SMI | ϕ | SMI | ϕ | SMI | ϕ |
| Stage I | .65 | .13 | .57 | .93 | .58 | .15 |
| Stage II | .52 | .002 | .5 | .17 | .5 | .002 |
| Stage III | .51 | .003 | .5 | .13 | .61 | .01 |
| Stage IV | .31 | 3.4 × 10⁻⁸ | .67 | .06 | .41 | .004 |

TABLE 17
Showing the SMI and ϕ Values for Other (American Indian/AK
Native, Asian/Pacific Islander) Race Groups for Age
Group [80-above)
OTHERS
Age: [80-Above)

| Treatment | | |
|---|---|---|
| C | R | C + R |

| Parameter | SMI | ϕ | SMI | ϕ | SMI | ϕ |
|---|---|---|---|---|---|---|
| Stage I | .7 | .42 | .69 | .67 | .84 | 3.19 |
| Stage II | .5 | .02 | .72 | .49 | .72 | .18 |
| Stage III | .6 | .18 | 1.75 | 1.1 | 1.26 | 1.96 |
| Stage IV | .42 | .0003 | .77 | .3 | 1.41 | 1.31 |

In Table 15, the “-” in Stage I, for age group [40-59) can imply that there are insufficient data points to calculate the SMI and ϕ values. From Table 8, there is only a single observation that falls under the category. Since any inference based on a single observation can be misleading, the SMI and ϕ values are not calculated for the specific group of patients. Under the age group [40-59) at Stage III, the patients who received only radiation therapy the SMI is 1.44, an indication that the failure intensity is increasing. Also, the same scenario can exist for the patients belonging to the age group [80-above) at Stage III who received only radiation (SMI=1.75), for the patients who received chemotherapy and radiation together (SMI=1.26), and for the patients at Stage IV who received chemotherapy and radiation together (SMI=1.41).

Conclusion: In the present disclosure, two aspects are disclosed: 1) Analytical Development in the subject area and 2) Data Analysis and Monitoring the survival Time of a specific group of patients. The Survival Monitoring Indicator (SMI) is defined and how it could be implemented in the survival data of pancreatic cancer patients is explained. Also, the Survival Monitoring Indicators (SMI) is calculated for all the all cancer stages, categorized by races and age three age groups. These SMI values can be used in deciding the mortality rate of patients as a function of time. Also, a criterion is derived for the relative change in intensity (RCI) based on SMI. The analytical process determines the behavior of RCI when any two SMI≤1 or ≤1 for any two specific groups of patients. Finally, the range of the study time t is determined based on the SGIF at) of two groups as a function of SMI. Our analytical method is useful for determination of the order of any two SGIFs ζ₁(t) and ζ₂(t) (which one is greater than other) based on the time range

$$( 0 , {δ}^{\frac{1}{( {SMI}_{2} - {SMI}_{1} )}} ] or [ {δ}^{\frac{1}{( {SMI}_{2} - {SMI}_{1} )}} , ∞ ) .$$

In our study, there are thirty-six groups of patients for each race, totaling (36×3=108) patient groups. A comparison of the SGIFs can be made between any two groups knowing the time range without actually computing the SGIFs. Conversely, if we don't have the information regarding the specific survival time t but we know two SGIFs ζᵢ(t) and ζⱼ(t) for any two specific groups i and j, (i≠j=1, 2, . . . , 108) out of 108 groups, we can estimate the interval for the specific time to death. The whole process can be summarized in an algorithmic form using the following steps:
1) Determine the specific two groups (i and j, i≠j=1, 2 , . . . , 108) as per requirement.
2) Determine the number of individuals nᵢ and nⱼ for the i^{th} and j^{th} groups, respectively.
3) Arrange the observations from the lowest to highest in each groups.
4) Determine the highest observations in each groups tₙᵢ and tₙⱼ.
5) Compute SMIᵢ and SMIⱼ using Equation (2-6).

6) Compute

$$δ = \frac{{SMI}_{i}}{{SMI}_{j}} \frac{{t}_{{n}_{j}}}{( {{n}_{j )}}^{1 / {SMI}_{j}}} \frac{( {{n}_{j )}}^{1 / {SMI}_{i}}}{{t}_{{n}_{j}}} .$$

7)

$${ζ}_{i} ( t ) \leq {ζ}_{j} ( t ) ↔ t ∈ [ {δ}^{\frac{1}{( {SMI}_{j} - {SMI}_{i} )}} , ∞ ) and$$

$${ζ}_{i} ( t ) \geq {ζ}_{j} ( t ) ↔ t ∈ ( 0 , {δ}^{\frac{1}{( {SMI}_{j} - {SMI}_{i} )}} ] ,$$

where ζᵢ(t) and ζⱼ(t) are the SGIs for i^{th} and j^{th} groups at time t. That is

$${SGF}_{1} ( t ) \leq S G {F}_{2} ( t ) ↔ t ∈ [ {δ}^{\frac{1}{( {SMI}_{2} - {SMI}_{1} )}} , ∞ ) and$$

$${SGF}_{1} ( t ) \geq S G {F}_{2} ( t ) ↔ t ∈ ( 0 , {δ}^{\frac{1}{( {SMI}_{2} - {SMI}_{1} )}} ] .$$

In some examples, the cancer disease stochastic model can be used for a patient to estimate a changing survival time due to a treatment option (e.g., using a medical website portal, or any suitable medical system). For example, as patients and their families attempt to assess treatment options and whether to undergo, forego, or cease treatment (for quality of life determinations), the foregoing techniques could be implemented via an app or software portal. A patient's changing survival time due to a given treatment could be provided based on a given treatment option. In addition, healthcare/pharmaceutical companies, healthcare professionals/doctors, health science researchers, cancer institutes and departments of public health, health policy makers, biomedical research institutes, government health organizations and/or any other suitable organization can use the cancer disease stochastic model for medical records, commercial products, research, or any other suitable purposes. For example, an electronic medical record system could indicate for a healthcare provider what the expected change (benefit or none) is in a patient's survival likelihood given a treatment, so as to give healthcare providers the ability to stop or change a treatment while there is still time for the patient.

### Example 3: A Modern Approach of Survival Methodologies: Cancer Patients Across Stages & Gender

Pancreatic cancer is one of the most fatal human cancers and continues to be a major unsolved health problem at the start of the 21st century. It has been estimated that this disease causes 30,000 deaths per year in the United States. It is the fourth leading cause of cancer death in the United States and leads to an estimated 227,000 deaths per year worldwide. The incidence and number of deaths caused by pancreatic tumors have been gradually increasing, even as incidence and mortality of other common cancers have been declining. Despite developments in detection and management of pancreatic cancer, only about 4% of patients will live 5 years after diagnosis. The normal pancreas consists of digestive enzyme-secreting acinar cells, bicarbonatesecreting ductal cells, centro-acinar cells that are the geographical transition between acinar and ductal cells, hormone-secreting endocrine islets and relatively inactive stellate cells. The majority of malignant neoplasms of the pancreas are adenocarcinomas. Rare pancreatic neoplasms include neuroendocrine tumors (which can secrete hormones such as insulin or glucagon) and acinar carcinomas (which can release digestive enzymes into the circulation).Specifically, ductal adenocarcinoma is the most common malignancy of the pancreas; this tumor (commonly referred to as pancreatic cancer) presents a substantial health problem, with an estimated 367,000 new cases diagnosed worldwide in 2015 and an associated 359,000 deaths in the same year. After the detection of pancreatic cancer, doctors usually perform some additional tests to understand better if cancer has been spread or the locations of spreading areas of the cancer. Imaging tests, such as a PET scan, help doctors identify the presence of cancerous growths. With these tests, doctors try to establish cancer's stage of a given patient with pancreatic cancer. Staging helps explicate the advancement of cancer. It also assists doctors in deciding treatment options. Once a diagnosis has been made, the doctor allocates the patient a stage based on the following test results: Stage I: Tumors exist solely in the pancreas. Stage II: Tumors have spread to adjacent abdominal tissues or lymph nodes. Stage III: The cancer has spread to major blood vessels and lymph nodes. Stage IV: Tumors have spread to other organs, such as the liver, lung, bone, etc.

Although in most of the cases, pancreatic cancer disease remains irremediable, most researches studying this type of cancer have focused on how to improve the survival times of patients diagnosed with pancreatic cancer at different cancer stages. The present disclosure presents a new parametric approach of survival analysis of patients diagnosed with Pancreatic Cancer. The unique probability distribution that characterizes the probabilistic behavior of the survival times can be found to obtain the survival function that is driven by the given data. In the present disclosure, the probability distribution that fits the survival times can be found and, the survival function of male and female patients in four different stages can be obtained.

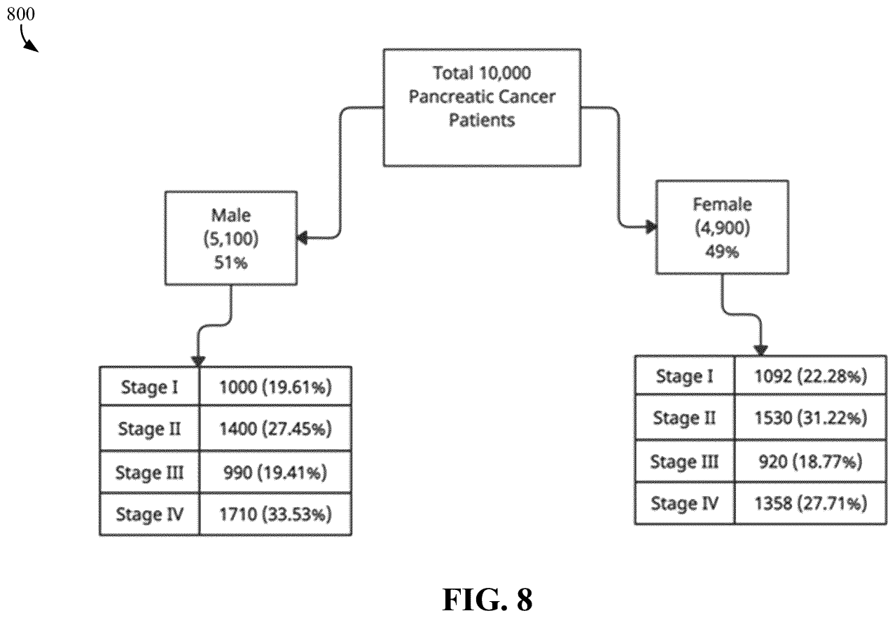

Methodology—Data Description: This section provides the data discussion, and investigate if there exist any significant difference between the male and female patients at any individual cancer stages. The data for the present disclosure has been extracted from the Surveillance, Epidemiology and End Results (SEER) database. The data contains information on patients diagnosed with pancreatic adenocarcinoma to identify the survival time (in months) and cause-specific death (deaths due to pancreatic cancer) for each patient. The survival time of patients is one of the factors used in all cancer research. It is desirable to evaluate the severity of cancer, which helps to decide the prognosis and help identify the correct treatment methods. Random samples of 10,000 patients diagnosed with pancreatic cancer including male and female are considered. A schematic diagram of the data used in this study with additional details is shown in FIG. 8. FIG. 8 shows pancreatic cancer data sorted by gender and stages. As the following schematic diagram illustrates, in our dataset, we have information on survival times regarding 5,100 male and 4,900 female patients diagnosed with pancreatic cancer. Before the parametric analysis of the survival times of patients is performed,

whether there is a difference in the true median survival times of genders, i.e., male and female patients in different stages of cancer can be investigated. For this purpose, the following hypothesis can be tested:

H₀: There is no statistically significant difference between the true median survival times of male (μ_{M}) and true median survival times of female (μ_{F}) patients at stage i.i=1, 2, 3, 4. That is μ_{M}=μ_{F}

### Vs.

H₁: Differences exist between male and female median survival times at stage i. That is, μ_{M}≠μ_{F}.

After the data is analyzed for male and female patients in each stages, the combined analysis can be performed for all stages, classified by gender. The following Table 18 illustrates the test results along with the p-values in different stages for male and female pancreatic cancer patients.

TABLE 18
Wilcoxon Test Results for Different Stages, Classified by Gender

| Stages | P-Values | Result |
|---|---|---|
| I | 0.75 | Difference does not Exist |
| II | 0.25 | Difference does not Exist |
| III | 0.84 | Difference does not Exist |
| IV | 0.001 | Difference Exists |

As, results of the above Table 18 suggests, there does not exist significant difference between the male and female pancreatic cancer patient survival times in stage I, stage II, and stage III. However, in stage IV, the difference is significant. In the next section, the parametric probability distributions and survival functions of the survival times of patients can be identified along with some descriptive statistics.

Descriptive Analysis of Pancreatic Cancer Patients in Different Stages-A Gender Based Classification: This section provides the stage-based descriptive analysis with graphical representation. In some examples, the histogram and probability density function (pdf) can be plotted to investigate the distribution of the survival times of patients in different stages. The probability distribution of the survival times can be right-skewed. The following Table 19 illustrates the different descriptive statistics for male and female patients in four different stages.

TABLE 19
Descriptive Statistics of Survival Time (in month) of Pancreatic
Cancer Patients Classified by Gender in Different Stages

| | | | Std. | | | Std. |
|---|---|---|---|---|---|---|
| Gender | Mean | Median | Dev. | Skewness | Kurtosis | Error |
| Combined | 30.6 | 20 | 31.5 | 1.33 | 1.14 | 0.76 |
| (Stage I) | | | | | | |
| Combined | 21.44 | 14 | 23.50 | 2.14 | 5.10 | .33 |
| (Stage II) | | | | | | |
| Combined | 16.92 | 8 | 14.71 | 3.73 | 20.01 | .37 |
| (Stage III) | | | | | | |
| Male | 6.7 | 3 | 12.73 | 4.78 | 30.44 | .18 |
| (Stage IV) | | | | | | |
| Female | 7.50 | 3.11 | 13.67 | 4.63 | 27.80 | .20 |
| (Stage IV) | | | | | | |

Now, an example probability distributions that drives the survival times of patients in different stages (I, II, III, and IV), classified by gender can be identified. There does not exist any significant difference between male and female survival times in stages I, II, and III. However, there is a difference in survival times of male and female patients in stage IV. In some examples, the best fits for each stages can be obtained, and their individual parameter estimates can be estimated. Identification of the most suitable probability distribution is desirable since it gives the better survival probability estimates for both male and female patients in each of the stages that is driven by the specific probability distribution. Once, the parameter estimates are obtained from the probability distributions at each of the stages, the probability density functions (pdfs), cumulative distribution functions (cdfs), and parametric survival function (S(t)) driven by the specific probability distribution for male and female patients individually can be obtained.

Parametric Analysis of Pancreatic Cancer Survival Time for Different Stages: In some examples, systems of different frequency curves based on transformations of the following form were proposed:

$$z = γ + δ f ( \frac{x - ζ}{λ} ) ,$$

where z is a unit Normal variable, ƒ is a function taking different forms S_{L}, S_{B}, and S_{U}. The data in Stage I follows Johnson S_{B} probability distribution with parameters γ (shape parameter), δ (shape parameter), ζ (location parameter), and λ (scale parameter). In Stage II, and Stage III, the data follows a generalized extreme value (GEV) probability distribution. In Stage IV, the data follows a generalized pareto (GP) probability distribution. This section discloses in detail about the parameter estimation procedure of generalized pareto (GP) probability distribution for overall survival times of the patients. The parameter estimation process of the Johnson SB distribution in Stage I can be described. Let T be a random variable denoting the survival times of patients in Stage I. Then, the pdf of T is given by,

$$f ( t ) = \frac{δ}{\sqrt{2 π}} \frac{λ}{( t - ζ ) ( λ + ζ - t )} exp [ - \frac{1}{2} {( γ + δ ln ( \frac{t - ζ}{λ + ζ - t} ) )}^{2} ] ,$$

$$ζ < t < ζ + λ ,$$

$$- ∞ < ζ < ∞ ,$$

$$λ > 0 ,$$

$$- ∞ < γ < ∞ ,$$

$$δ > 0$$

From the data, the extreme order statistics tₘᵢₙ and tₘₐₓ can be determined. In some examples, in Stage I, tₘᵢₙ=0, and tₘₐₓ=155. Since, ζ, and λ are the location and spread parameters respectively, \hat{ζ}=tₘᵢₙ=0 (since, the minimum value of the survival time t is 0), and \hat{λ}=(tₘₐₓ−tₘᵢₙ)=(155−0)=155=tₘₐₓ.

Given the estimated values, \hat{ζ}and \hat{λ}, the following transformation can be performed, that is, the values of tᵢ are transformed to:

$${f}_{i} = ln ( \frac{{t}_{i} - \hat{ζ}}{\overline{λ} + \hat{ζ} - {t}_{i}} ) .$$

The estimates of the other parameters \hat{γ}, and \hat{δ}, and take the following form:

$$\hat{γ} = - \frac{\overline{f}}{{S}_{f}} and \hat{δ} = \frac{1}{{S}_{f}} , where$$

$$\overline{f} = \frac{{\sum }_{i} {f}_{i}}{n} and {S}_{f} = \sqrt{\frac{{\sum }_{i} {( {f}_{i} - \overline{f} )}^{2}}{n}} .$$

The validity of the model assumptions can be justified using the goodness of fit tests. A Johnson S_{B} probability distribution can be fitted to the wind speed data, and Kolmogorov-Smirnov (K-S) and Anderson-Darling (A-D) tests can be used to justify goodness of fit assumptions. The same approach using Kolmogorov-Smirnov (K-S), Anderson-Darling (A-D), and Cram'er-von Mises (CVM) goodness of fit tests can be used. The following Table 20 provides the goodness of fit tests results along with the p-values for all probability distributions in the four different stages.

TABLE 20
Goodness of Test for Four Stages

| Stages | Gender | Prob. Distribution | GOF Tests | p-Values |
|---|---|---|---|---|
| I | Combined | Johnson S_{B} | A-D | .11 |
| | | | K-S | .13 |
| II | Combined | GEV | A-D | .27 |
| | | | K-S | .21 |
| III | Combined | GEV | A-D | .09 |
| | | | K-S | .1 |
| IV | Male | GPD | CVM | .22 |
| | | | K-S | .18 |
| IV | Female | GPD | CVM | .19 |
| | | | K-S | .17 |

As the p-values shown in Table 21 of the given data, the fact that the observations (survival times) follow the specified probability distributions in each of the four stages can be considered. The following Table 21, provides the specific probability distributions in each stages and their individual parameter estimates (approximate), classified by gender.

TABLE 21
Probability Distributions and Parameter Estimates of Survival Time
(in month) of Pancreatic Cancer Patients for Different Stages

| | | Probability | |
|---|---|---|---|
| Stages | Gender | Distributions | Parameter Estimates |
| I | Combined | 4-P Johnson S_{B} | \hat{γ} = 1.2, \hat{δ} = 0.62, |
| | | | \hat{λ} = 155, \hat{ζ} = 0 |
| II | Combined | Gen. Extreme | \hat{μ} = 10.18, \hat{σ} = 10.83, |
| | | Value (GEV) | \hat{k} = 0.32 |
| III | Combined | Gen. Extreme | \hat{μ} = 5.54, \hat{σ} = 6.07, |
| | | Value (GEV) | \hat{k} = 0.37 |
| IV | Male | 3-P Gen. | \hat{μ} = 0, \hat{σ} = 4.12, |
| | | Pareto (GP) | \hat{k} = 0.25 |
| IV | Female | 3-P Gen. | \hat{μ} = 0, \hat{σ} = 4.63, |
| | | Pareto (GP) | \hat{k} = 0.41 |

The following Table 22 illustrates the analytical forms of the probability density functions of male and female patients for the different stages, with the parametric estimates.

TABLE 22
Probability Distributions with their Parameter Estimates of the Survival Times (in months) of
Pancreatic Cancer Patients Classified by Gender for Different Stages.

| Gender | Analytical Forms |
|---|---|
| Combined (I) | $f ( t ) = \frac{\text{.62}}{\sqrt{2 n}} \frac{155}{t ( 155 - t )} exp [ - \frac{1}{2} {( 1.2 + \text{.62} ln ( \frac{t}{155 - t} ) )}^{2} ]$ |
| Combined (II) | $f ( t ) = \frac{1}{10.83} exp [ - {( 1 - \text{.32} ( \frac{t - 10.18}{10.83} ) )}^{3.125} ] {( 1 - \text{.32} ( \frac{t - 10.18}{10.83} ) )}^{2.125}$ |
| | $f ( t ) = \frac{1}{6.07} exp [ - {( 1 - \text{.32} ( \frac{t - 5.54}{6.07} ) )}^{2.7} ] {( 1 - \text{.32} ( \frac{t - 5.54}{6.07} ) )}^{1.7}$ |
| | $f ( t ) = {\frac{1}{4.12} [ 1 - \text{.25} ( \frac{t}{4.12} ) ]}^{3}$ |
| | $f ( t ) = {\frac{1}{4.63} [ 1 - \text{.34} ( \frac{t}{4.63} ) ]}^{1.44}$ |

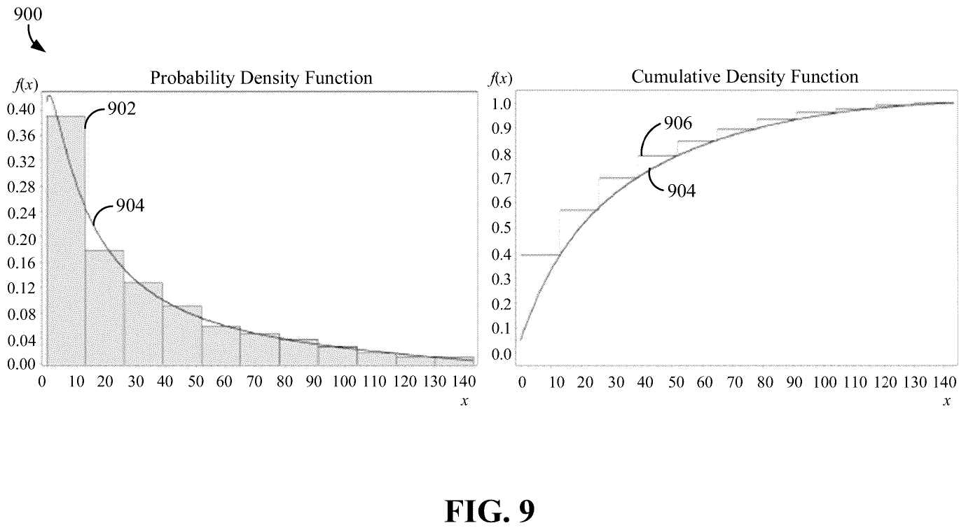

FIG. 9 illustrates the probability density function (pdf) and cumulative distribution function (cdf) of the patients in stage I. FIG. 9 shows the histogram 902 and the pdf plot of stage I with Johnson SB 904 and the sample 906 and the cdf plot of stage I with Johnson SB 904.

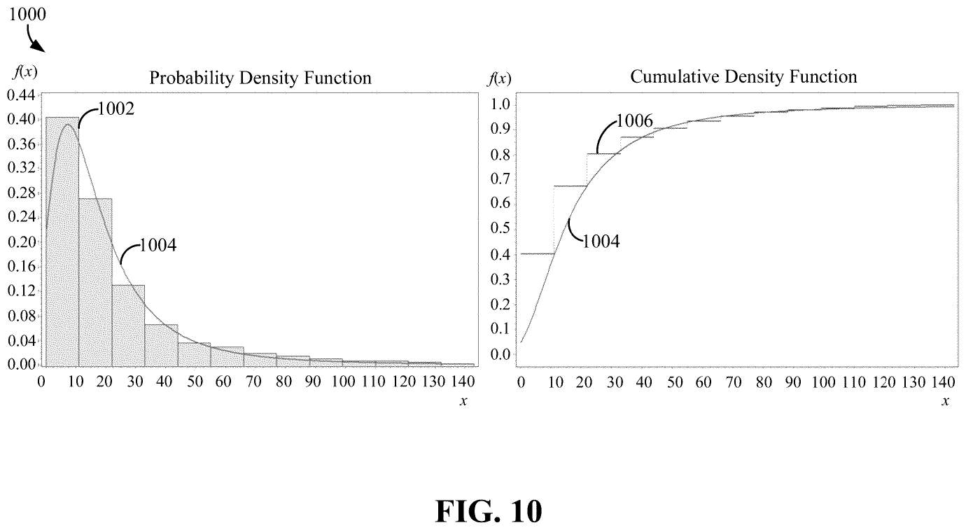

FIG. 10 shows the histogram 1002, pdf and cdf plots of stage II pancreatic cancer patients. In FIG. 10, the pdf plots include the histogram 1002 and generated extreme values 1004 while the cdf plots include the generated extreme values 1004 and the samples 1006.

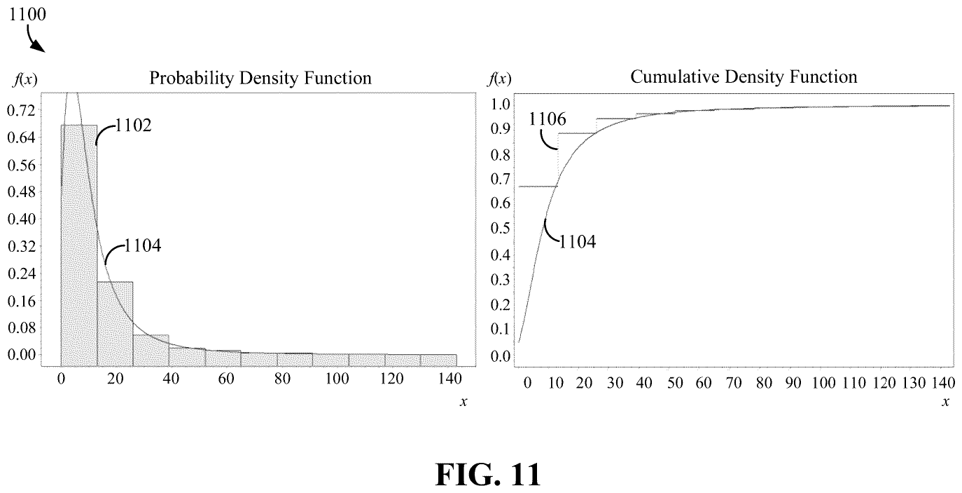

FIG. 11 shows the histogram 1102, pdf and cdf plots of stage III pancreatic cancer patients. In FIG. 11, the pdf plots include the histogram 1102 and generated extreme values 1104 while the cdf plots include the generated extreme values 1104 and the samples 1106.

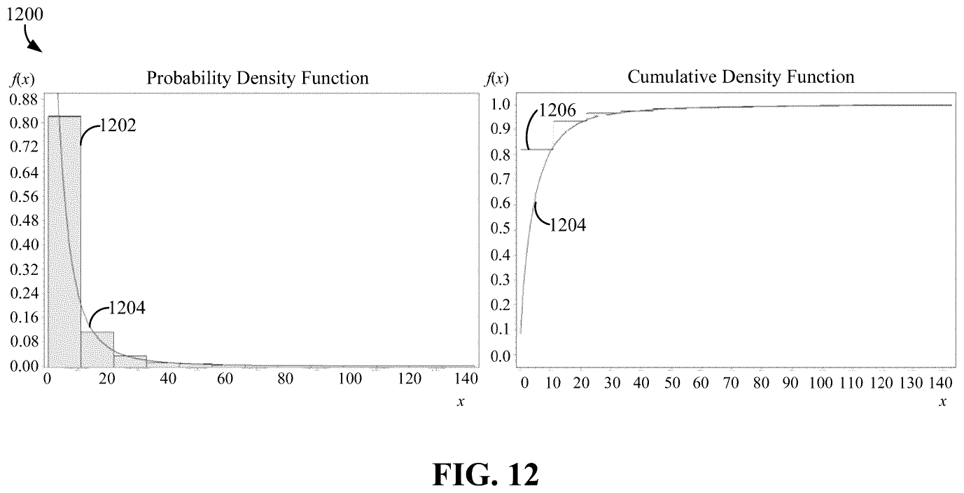

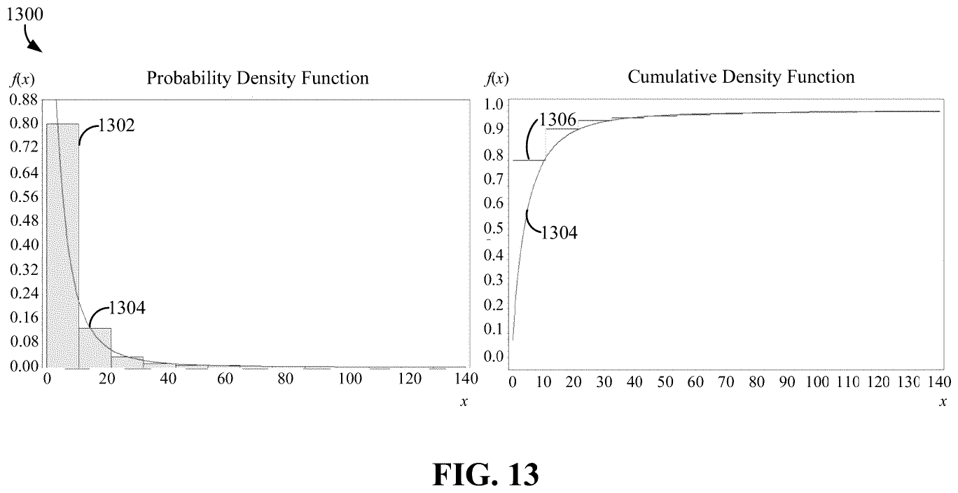

FIG. 12 describes the histogram, pdf, and cdf of male and female survival time respectively in stage IV while FIG. 13 describes the histogram, pdf, and cdf of female survival time respectively in stage IV. In FIGS. 12 and 13, the pdf plots include the histogram 1202, 1302 and generated extreme values 1204, 1304 while the cdf plots include the generated extreme values 1204, 1304 and the samples 1206, 1306.

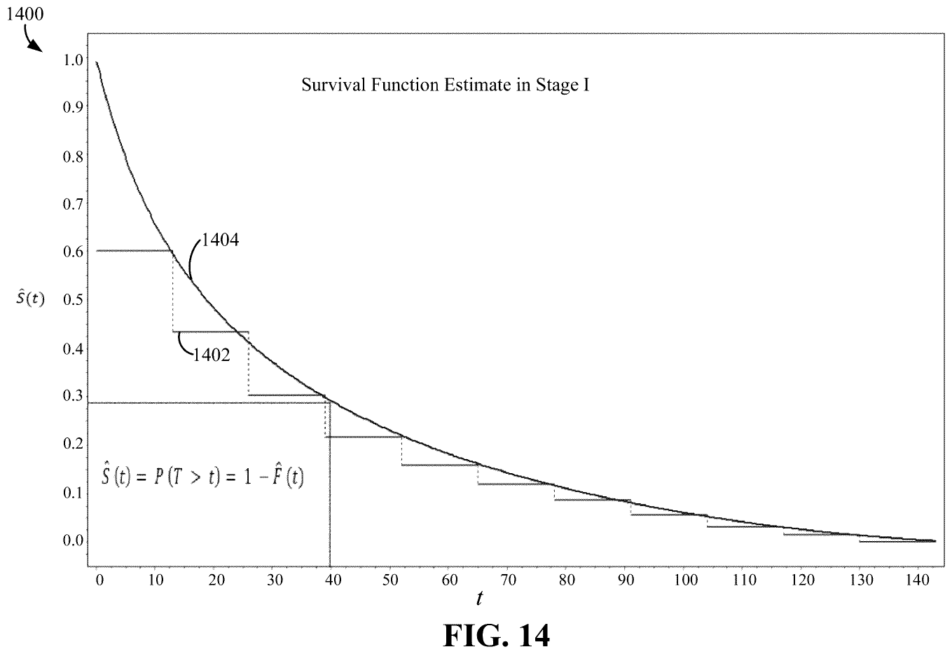

Parametric Survival Analysis for Different Stages: Once the analytical structures of the survival times of patients in different stages are driven by different parametric probability distributions, the survival function S(t) can be expressed analytically as a function of the cumulative distribution function (cdf). In some examples, the analytical forms of the survival functions for the four different stages can be expressed with respect to the analytical forms given in Table 22. The estimate of the parametric survival function of patients diagnosed with pancreatic cancer in Stage I can be given by:

$$\begin{matrix}{\hat{S}}_{I} ( t ; \hat{ζ} , \hat{λ} , \hat{γ} , \hat{δ} ) = 1 - {\hat{F}}_{I} ( t ; \hat{ζ} , \hat{λ} , \hat{γ} , \hat{δ} ) \\ = 1 - Φ [ \hat{γ} + \hat{δ} ln ( \frac{t - \hat{ζ}}{\hat{λ} - t + \hat{ζ}} ) ] \\ = 1 - Φ [ 1.2 + \text{.62} ln ( \frac{t}{155 - t} ) ] , t \geq 0.\end{matrix} \qquad ( 3 - 1 )$$

where Φ(·) is the cdf of a standard normal probability distribution and \hat{F}_{I} (t; \hat{ζ}, \hat{λ}, \hat{γ}, \hat{δ}) can be the estimated cdf of Johnson S_{B} probability distribution. The survival function Ŝ(·;·) can be used to estimate the probability that a patient diagnosed with pancreatic cancer would survive beyond time t, which is denoted by P (T≥t). For example, the probability that a male patient diagnosed with pancreatic cancer would survive beyond 30 months can be computed. For example, for t=40 in equation (3-1), the probability is 0.29 approximately. Thus, a randomly chosen patient classified in Stage I with pancreatic cancer has a 29% chance of survival beyond 40 months, as shown by FIG. 14. FIG. 14 shows a parametric survival plot of pancreatic cancer patients in Stage I. In FIG. 14, the survival function estimate in Stage I includes the samples 1402 and the Johnson SB 1404.

Similarly, the estimate of parametric survival function of patients, driven by GEV probability distribution function diagnosed with pancreatic cancer in Stage II is given by:

$$\begin{matrix}{\hat{S}}_{II} ( t ; \hat{μ} , \hat{σ} , \hat{k} ) = 1 - {\hat{F}}_{II} ( t ; \hat{μ} , \hat{σ} , \hat{k} ) \\ {= 1 - exp [ - ( 1 - k \frac{t - \hat{μ}}{\hat{σ}} ) )}^{\frac{1}{k}} ] \\ = 1 - exp [ - {( 1 - \text{.32} ( \frac{t - 10.18}{10.83} ) )}^{\frac{1}{\text{.32}}} ] , t \geq 10.18\end{matrix} \qquad ( 3 - 2 )$$

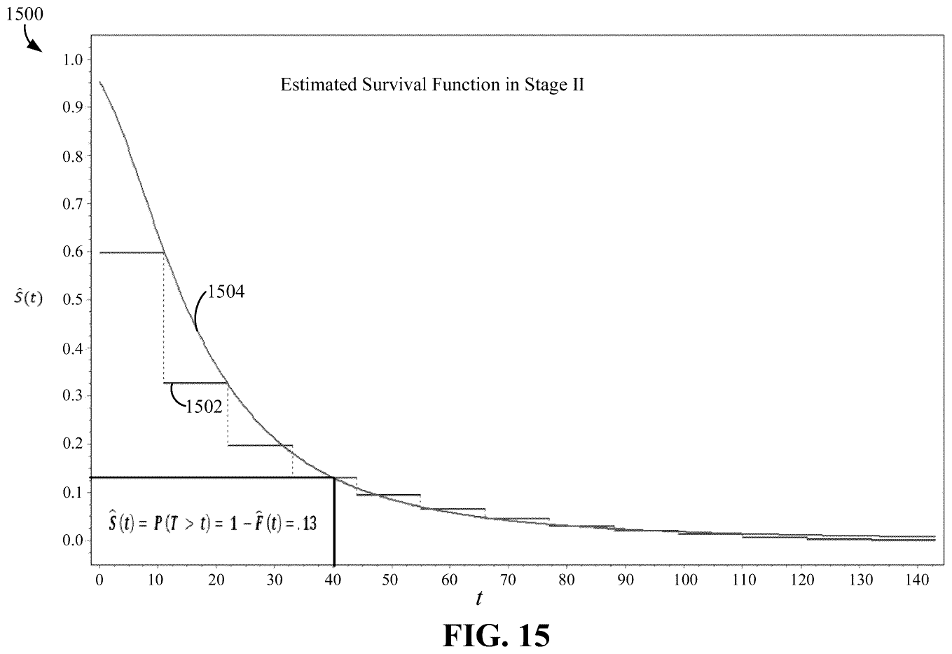

FIG. 15 shows a survival plot for Stage II patients. In FIG. 15, patients in stage II have comparatively lower survival probability than stage I patients, which is quite natural. With reference to the last example, the survival probability can be predicted as 13% for a Stage II patient, surviving beyond 40 months. In FIG. 15, the estimated survival plot in Stage II includes the samples 1502 and the generated extreme values 1504.

Now we proceed to express the GEV in analytical form for the stage III patients in a similar manner. The survival function at stage III can be given by:

$$\begin{matrix}{\hat{S}}_{III} ( t ; \hat{μ} , \hat{σ} , \hat{k} ) = 1 - {\hat{F}}_{III} ( t ; \hat{μ} , \hat{σ} , \hat{k} ) \\ = 1 - exp [ - {( 1 - k ( \frac{t - \hat{μ}}{\hat{σ}} ) )}^{\frac{1}{k}} ] \\ = 1 - exp [ - {( 1 - \text{.37} ( \frac{t - 5.54}{6.07} ) )}^{\frac{1}{\text{.37}}} ] , t \geq 5.54\end{matrix} \qquad ( 3 - 3 )$$

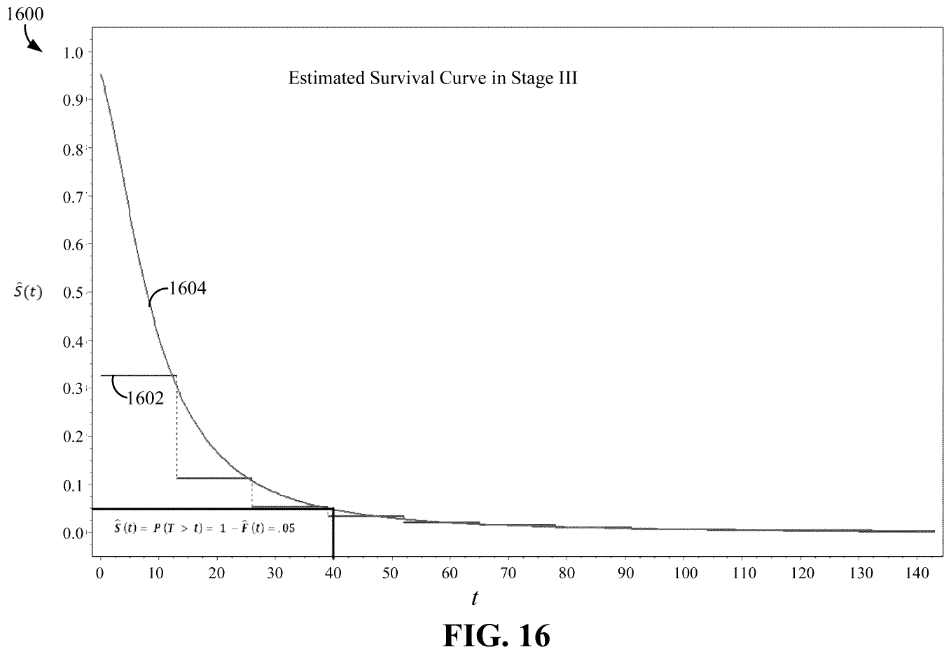

FIG. 16 shows that the survival probability is decreasing and it is approximately 5% for a randomly chosen patient who will survive beyond t=40 months after the patient is diagnosed with pancreatic cancer, Stage III. In FIG. 16, the estimated survival plot in Stage III includes the samples 1602 and the generated extreme values 1604.

Results from Table 18 suggest that there is a significant difference between the true mean survival times of stage IV patients, classified by gender. Thus, the analytical forms of the survival times for male and female patients can be separately expressed at Stage IV. The parametric survival function, driven by GPD, at stage IV male patients can be expressed as:

$$\begin{matrix}{\hat{S}}_{IV} ( t ; \hat{μ} , \hat{σ} , \hat{k} ) = 1 - {\hat{F}}_{IV} ( t ; \hat{μ} , \hat{σ} , \hat{k} ) \\ = 1 - [ 1 - ( {[ 1 + \hat{k} ( \frac{t - \hat{μ}}{\hat{σ}} ) ]}^{- \frac{1}{k}} ) ] \\ = ( {[ 1 + \text{.25} ( \frac{t}{4.12} ) ]}^{- \frac{1}{\text{.25}}} ) , t \geq 0.\end{matrix} \qquad ( 3 - 4 )$$

Similarly, the parametric survival function, driven by GDP, at stage IV female patients can be given by:

$$\begin{matrix}{\hat{S}}_{IV} ( t ; \hat{μ} , \hat{σ} , \hat{k} ) = 1 - {\hat{F}}_{IV} ( t ; \hat{μ} , \hat{σ} , \hat{k} ) \\ = 1 - [ 1 - ( {[ 1 + \hat{k} ( \frac{t - \hat{μ}}{\hat{σ}} ) ]}^{- \frac{1}{k}} ) ] \\ = ( {[ 1 + \text{.41} ( \frac{t}{4.63} ) ]}^{- \frac{1}{\text{.41}}} ) , t \geq 0.\end{matrix} \qquad ( 3 - 5 )$$

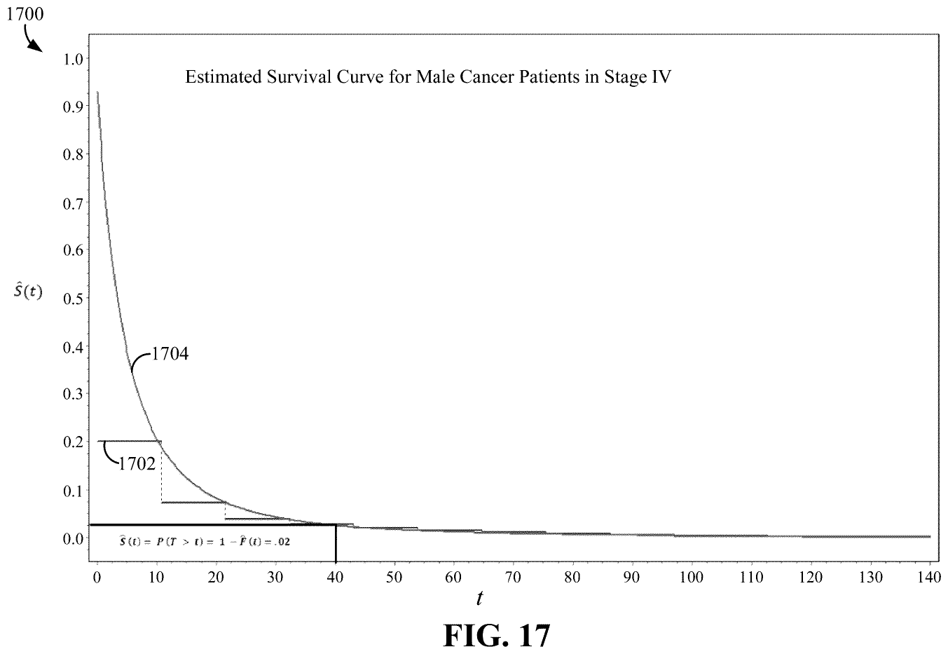

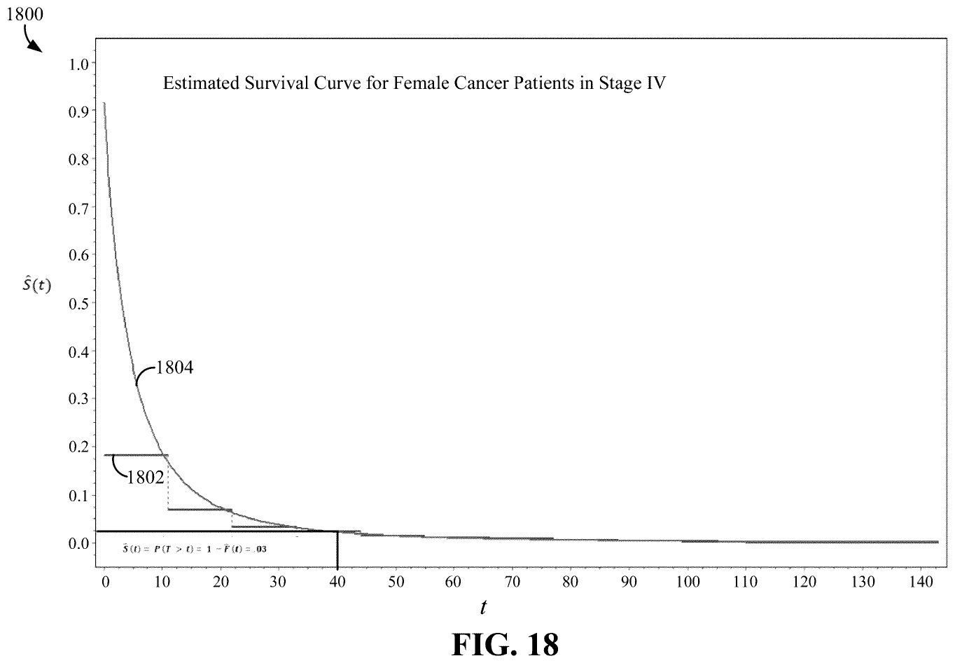

FIGS. 17 and 18 show that the survival probabilities are extremely low (2% for male patients and 3% for female patients) for surviving beyond t=40 months after the diagnosis at Stage IV. In FIGS. 17 and 18, the parametric survival plots can include samples 1702, 1802 and the generated Pareto values 1704, 1804.

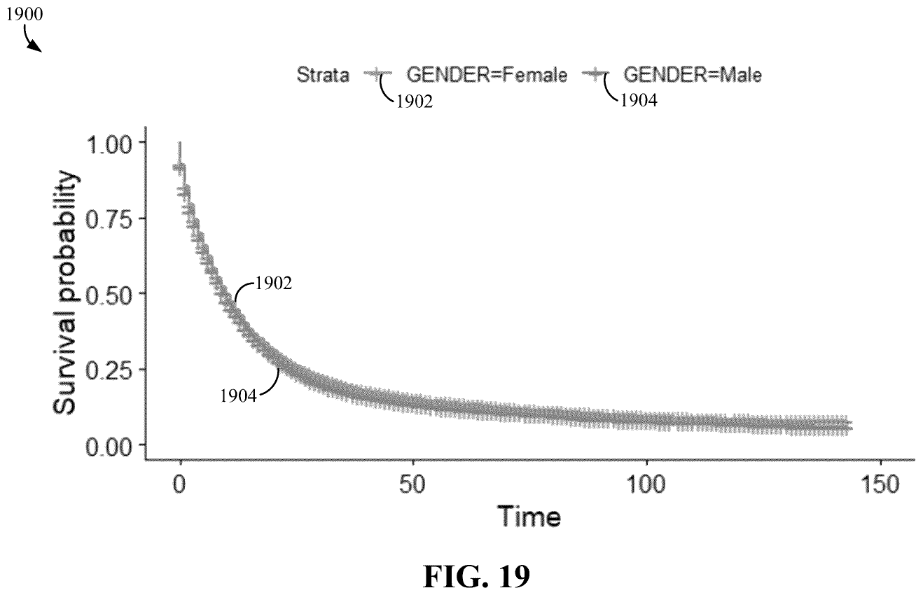

Parametric Analysis of The Survival Times of Patients with Pancreatic Cancer-A Combined Analysis: The parametric analytical forms of the survival times of patients in different stages are described above. Also, the survival functions of patients in different stages are computed. In addition, inventors found that there is no significant different in the survival times of male and female patients except stage IV. Now, the same process can be performed for the combined data, irrespective of stage. At first, it can be checked to see if there exists significant difference between the true mean survival times of male and female pancreatic cancer patients. Inventors found that there is insufficient sample evidence to reject the hypothesis that the distribution of mean survival times between the male and female patients diagnosed with pancreatic cancer is the same. FIG. 19 illustrates the behavior of overall survival curves of male and female patients. As shown in FIG. 19, the survival curve of males 1904 and the survival curve of females 1902 are almost identical which implies that they exhibit similar characteristics.

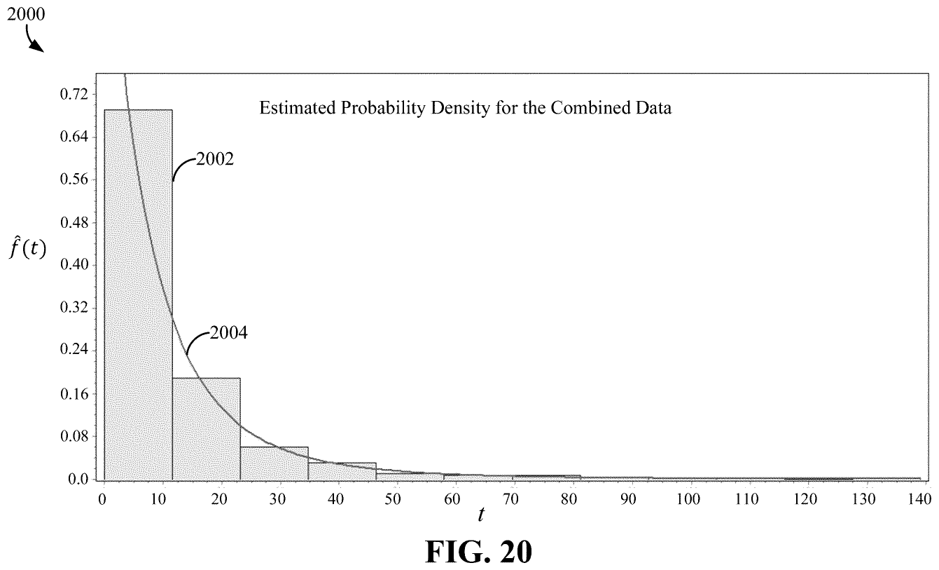

Descriptive Statistics of the Survival Times of Pancreatic Cancer Patients: In this section, the combined survival data is descriptively analyzed. In some examples, the histogram and probability density function (pdf) are plotted to investigate the probability distribution of the survival times of pancreatic cancer patients as shown in FIG. 20. FIG. 20 shows a histogram and probability density of survival times of combined pancreatic cancer patients. In the FIG. 20, the probability distribution of the overall survival time is right-skewed. Table 23 displays the descriptive statistics of the overall survival times for pancreatic cancer patients. In Table 23, the mean (average) survival times patients diagnosed with pancreatic cancer is 18 months. It implies that a randomly chosen patient diagnosed with pancreatic cancer is expected to survive for 18 months on an average. Also, the median survival time is nine months, which implies that the probability/chance of survival of a male or female patient beyond nine months, is approximately 50%. A negative (less than zero) skewed value implies that data distribution is left or negatively skewed, and a positive skewed value suggests that data is right or positive skewed. Thus, the positive skewed value of 3.07, as shown in Table 23, for patients diagnosed with pancreatic cancer, is further evidence to support the right-skewed behavior of the data, as shown in FIG. 20. Kurtosis can support the assessment of the extreme values of the data, and its positive value illustrates a leptokurtic behavior of the distribution. In contrast, a negative value shows a platykurtic behavior of the data distribution. Thus, the kurtosis value of 12.67 in Table 23 can attest to the leptokurtic behavior of the survival data. Table 23 illustrates the different descriptive statistics for survival times of all patients combined, diagnosed with pancreatic cancer.

TABLE 23
Descriptive Statistics of Survival Time (in month)
of Overall Pancreatic Cancer Patients Classified.

| Descriptive Statistics | Measures |
|---|---|
| Mean | 10.87 |
| Median | 6 |
| Std. Dev. | 14.63 |
| Skewness | 3.07 |
| Kurtosis | 12.67 |
| Std. Error | .24 |

Three Parameter Generalized Pareto (GP) Probability Estimation of The Survival Times of Patients with Pancreatic Cancer: A parametric analysis of the survival times of patients diagnosed can be performed with cancer (e.g., pancreatic cancer) to identify the underlying probability distribution, which characterizes the probabilistic behavior of the survival times of patients (both genders). In the attempt to obtain the best-fitted probability distribution, a number of classical distributions were tested to fit the data. The inventors used the Anderson-Darling test and Cram'er-von Mises test to identify the best probability distribution function that characterizes the probabilistic behavior of the survival times patients. Also, the expected survival times and median survival times that is driven by the best fitted probability distribution are estimated. The best-fitted probability distribution that characterizes the probabilistic behavior of the survival times of the male and female patients accurately is the three parameter (3-P) Generalized Pareto (GP) probability distribution. Table 24 below shows the goodness of fit (GOF) results of the 3-P GPD distribution.

TABLE 24
Goodness-of-fit Test of the GPD of the
Survival Times of Male and Female

| Statistical Tests | P-Values Male | P-Values Female |
|---|---|---|
| Kolmogorov-Smirnov | 0.27 | .38 |
| Cram'er-von Mises | 0.22 | .18 |

The above results show that the null hypothesis that the subject data (survival times for males and females) follow a GP probability distribution can be considered. In this section, the probability density function (pdf) of the Generalized Pareto distribution and the statistical approach to obtain approximate estimates of its parameters are defined. In the domain of probability theory and statistics, the Generalized Pareto distribution (GPD) is a family of continuous probability distributions developed based on the extreme value theory. The GPD is a generalization of the Pareto distribution (PD). In some examples, let T be a random variable following GPD with location parameter μ, scale parameter σ>0 and shape parameter k. That is, T˜GPD (μ, σ, k) with the domain

$$μ \leq x \leq μ - \frac{σ}{k} ,$$

when k<0 and μ≤t<∞, when k≥0. Then, the probability density function (pdf) of T can be given as follows:

$${f}_{GPD} ( t ; μ , σ , k ) = \{ \begin{matrix}\frac{1}{σ} ( {[ 1 + k ( \frac{t - μ}{σ} ) ]}^{- \frac{1}{k} - 1} ) , & k \neq 0 \\ \frac{1}{σ} exp ( - \frac{( t - μ )}{σ} ) , & k = 0\end{matrix} \qquad ( 3 - 6 )$$

The corresponding cumulative distribution function (cdf) can be given as follows:

$${F}_{GPD} ( t ; μ , σ , k ) = \{ \begin{matrix}1 - ( {[ 1 + k ( \frac{t - μ}{σ} ) ]}^{- \frac{1}{k}} ) , & k \neq 0 \\ 1 - exp ( - \frac{( t - μ )}{σ} ) , & k = 0\end{matrix} \qquad ( 3 - 7 )$$

There are several methods for estimating the parameters μ, σ, and k of the GP distribution. Some of these methods include elemental percentile method (EPM) and an algorithm for computing the maximum likelihood estimation (MLE) of the parameters of the GPD. In other examples, a parameter and quantile estimation mechanism can be derived based on Probability-weighted moments (PWM). In further examples, an improved maximum likelihood estimation using the empirical Bayesian method can overcome the non-existence problem of the PWM estimator. In further examples, a more efficient optimization algorithm for estimators of the GPD parameters can be used where the proposed estimators are defined for all possible values of the parameters. In some examples, the performance of the estimators can be better than the method of moments (MOM) and Probability-Weighted Moments (PWM) estimates. In further examples, a GP parameter estimation method can be used for censored data and validated their results using sensitivity and specificity test. In even further examples, a parameter estimation method using the principle of maximum entropy (POME) for 3-P GPD can be used. Any method described above can be chosen for the parameter estimation purpose. The parameter estimation procedure of 3-P GPD by PWM method is disclosed below.

Parameter Estimation of 3-P GPD Using the Method of Probability-Weighted Moments (PWM): The probability-weighted moments (PWM) of a random variable T with cumulative distribution function F(t)=P(T≤t) can be given by:

M_{p,r,s}=E[T^{p}{F(t)}^{r}{1−F(t)}^{s}], (3-8)

where p, r, and s are real numbers. Probability-weighted moments can be expressed as a function of the inverse distribution function F⁻¹(t)=t(F) in closed form by:

M_{p,r,s}=∫₀¹{f(F)}^{p}F^{T}{1−F}^{s}] (3-9)

The two special cases of M_{p,r,s} can be:

αₛ=M_{1,0,s}=E[T{1−F(t)}^{s}], s=0, 1, 2, . . . )

and

βᵣ=M_{1,r,0}=E[T{F(t)}^{r}], (r=0, 1, 2, . . . ), (3-10)

where T inside the E[·] can be the inverse distribution of T, denoted by t(F). To estimate the parameters of GPD , αₛ=M_{1,0,s}=E[T{1−F(t)}^{s}] can be used.

From Equation (3-7), T can be solved to obtain the inverse cdf, t(F). The inverse distribution function can be given by:

$$t ( F ) = \{ \begin{matrix}μ + \frac{σ}{k} \{ 1 - ( 1 - {F}^{k} ) \} & if k \neq 0 \\ μ - σ log ( 1 - F ) & if k = 0\end{matrix} . \qquad ( 3 - 11 )$$

The analytical form of αₛ for the 3-P GPD can be given as follows. Using expressions (3-10) and (3-11). From (3-10), Equation (3-12) can be expressed as:

$$\begin{matrix}{α}_{s} = {M}_{1 , 0 , s} \\ = {\int }_{0}^{1} [ μ + \frac{σ}{k} \{ 1 - ( 1 - {F}^{k} ) \} ] [ 1 - {F}^{s} ] dF \\ = \begin{matrix}\frac{1}{s + 1} ( μ + \frac{σ}{k} ) - \frac{σ}{k} ( \frac{1}{k + s + 1} ) , & ( s = 0 , 1 , 2 , … )\end{matrix}\end{matrix} . \qquad ( 3 - 12 )$$

Thus, for k≠0, the probability-weighted moments (PWM) of the 3-P GP distribution can be given by (3-12). In Equation (3-12), by substituting s=0, r=1, and r=2, explicit expressions of α₀, α₁, and α₂ in terms of μ, σ and k can be obtained. That is,

$${α}_{0} = ( μ + \frac{σ}{k} ) - \frac{σ}{k} ( \frac{1}{k + 1} ) , \qquad ( 3 - 13 )$$

$${α}_{1} = \frac{1}{2} ( μ + \frac{σ}{k} ) - \frac{σ}{k} ( \frac{1}{k + 2} ) , and \qquad ( 3 - 14 )$$

$${α}_{2} = \frac{1}{3} ( μ + \frac{σ}{k} ) - \frac{σ}{k} ( \frac{1}{k + 3} ) . \qquad ( 3 - 15 )$$

The PWM estimates of the parameters (\hat{μ}, \hat{σ}, \hat{k}) can be obtained by solving the equations (13), (14) and (15) for μ, σ, and k. After solving the above three equations, the explicit expressions of the PWM estimates can be obtained as follow:

$$\hat{k} = \frac{{α}_{0} - 8 {α}_{1} - 9 {α}_{2}}{- {α}_{0} + 4 {α}_{1} - 3 {α}_{2}} . \qquad ( 3 - 16 )$$

$$\hat{σ} = \frac{( {α}_{0} - 2 {α}_{1} ) ( {α}_{0} - 3 {α}_{2} ) ( - 4 {α}_{1} + 6 {α}_{2} )}{{( - {α}_{0} + 4 {α}_{1} - 3 {α}_{2} )}^{2}} . \qquad ( 3 - 17 )$$

$$\hat{μ} = \frac{2 {α}_{0} {α}_{1} - 6 {α}_{0} {α}_{2} + 6 {α}_{1} {α}_{2}}{- {α}_{0} + 4 {α}_{1} - 3 {α}_{2}} . \qquad ( 3 - 18 )$$

Table 25 below shows the approximate parameter estimates of survival times driven by 3-P GP probability distribution.

TABLE 25
Parameter Estimates of 3-P GP Probability Distribution
of the Survival Times of Pancreatic Cancer Patients

| Estimates | Measures |
|---|---|
| Location (\hat{μ}) | .65 |
| Scale (\hat{σ}) | 8.9 |
| Shape (\hat{k}) | 0.22 |

In some examples, substituting the parameter estimates of μ, σ, k in Equation (3-6) can be used to obtain the analytical form of the probability density function (pdf) of patients' survival times. The analytical form of the GP probability density function (pdf) for combined pancreatic cancer survival time can be given by:

$$\begin{matrix}{f}_{Combined} ( t ) = {\frac{1}{8.9} [ 1 + \text{.22} ( \frac{t - \text{.22}}{8.9} ) ]}^{- 5.54} , & t \geq \text{.22} .\end{matrix} \qquad ( 3 - 19 )$$

The above probability density function can characterize the probabilistic behavior of the overall survival times of male and female patients with pancreatic cancer.

In further examples, the expected survival times E(T) of patients driven by GP probability distribution can be calculated. Using estimates given in Table 25, the expectations and median survival times for the patients that follow GPD (0.65, 8.9, 0.22) distribution can be found.

The expected value of a random variable T following GPD (μ, σ, k) can be given by:

$$\begin{matrix}E ( T ) = \hat{μ} + \frac{\hat{σ}}{1 - \hat{k}} , & \hat{k} < 1.\end{matrix} \qquad ( 3 - 20 )$$

Using Equation (3-20), the expected survival time for pancreatic cancer patients following GPD (0.65, 8.9, 0.22) can be

$$E ( T ) = \text{.65} + \frac{8.9}{1 - \text{.22}} = 12.06 months .$$

The median of the survival time T of GDP (μ, σ, k) can be given by:

$${Med}_{GPD} ( t ; μ , σ , k ) = \hat{μ} + \frac{\hat{σ} ( {2}^{\hat{k}} - 1 )}{\hat{k}} \qquad ( 3 - 21 )$$

From Equation (3-21), the overall median survival times of male and female pancreatic patients together is given by:

$$Med ( T ) = \text{.65} + \frac{8.9 ( {2}^{\text{.22}} - 1 )}{\text{.22}} = 7.31 months .$$

Once the analytical forms of the pdf are obtained, the cumulative distribution functions (cdf) of the random variable T can be obtained. The analytical form of the GPD cdf can be given by:

$$\begin{matrix}{F}_{Combined} ( t ) = 1 - {[ 1 + \text{.22} ( \frac{t - \text{.65}}{8.9} ) ]}^{- 4.54} , & t \geq \text{.65} .\end{matrix} \qquad ( 3 - 22 )$$

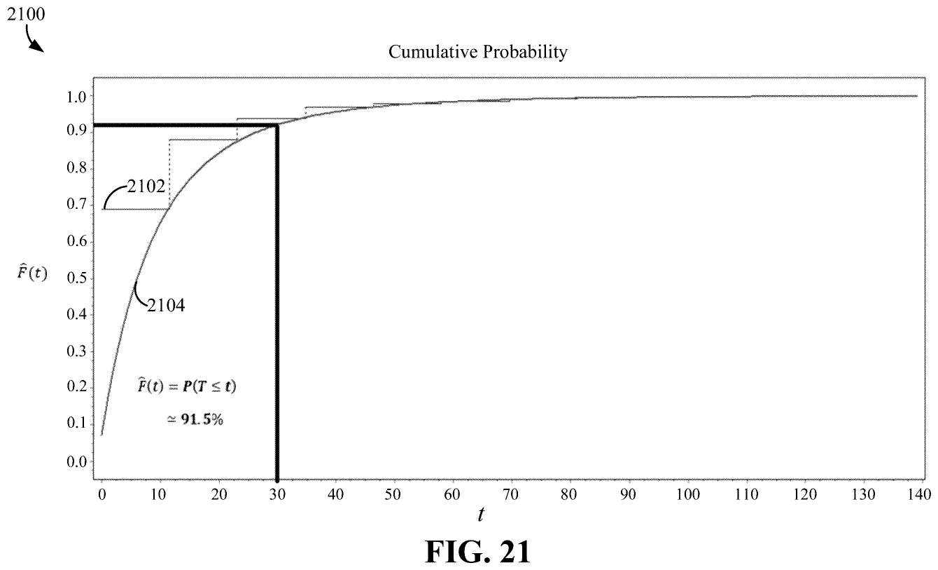

FIG. 21 illustrates the cdf plot of the overall survival times. As FIG. 21 illustrates, the cdf plot is helpful to estimate the probabilities that a certain male or female patient diagnosed with pancreatic cancer will survive up to a specific point of time. For example, from FIG. 21, the probability that a randomly diagnosed patient will survive up to time t=30 months is approximately 91.5%. The parametric survival analysis of the overall survival times of pancreatic cancer patients is presented below.

Parametric Survival Analysis: Estimation of a parametric survival function can be a process to evaluate the survival probabilities of male or female pancreatic cancer patients as a function of the survival time. Inventors have determined the cdf of the survival times for patients diagnosed with pancreatic cancer patients in Equation (3-22). Now, the survival function S(t) can be estimated. Thus, the parametric survival function of patients, irrespective of stages, diagnosed with pancreatic cancer can be given by,

$$\begin{matrix}\hat{S} ( t ; \hat{μ} , \hat{σ} , \hat{k} ) = 1 - \hat{F} ( t ; \hat{μ} , \hat{σ} , \hat{k} ) \\ = \begin{matrix}[ 1 + \text{.22} {( \frac{t - \text{.65}}{8.9} )}^{- 4.54} ] , & t \geq \text{.65}\end{matrix}\end{matrix} . \qquad ( 3 - 23 )$$

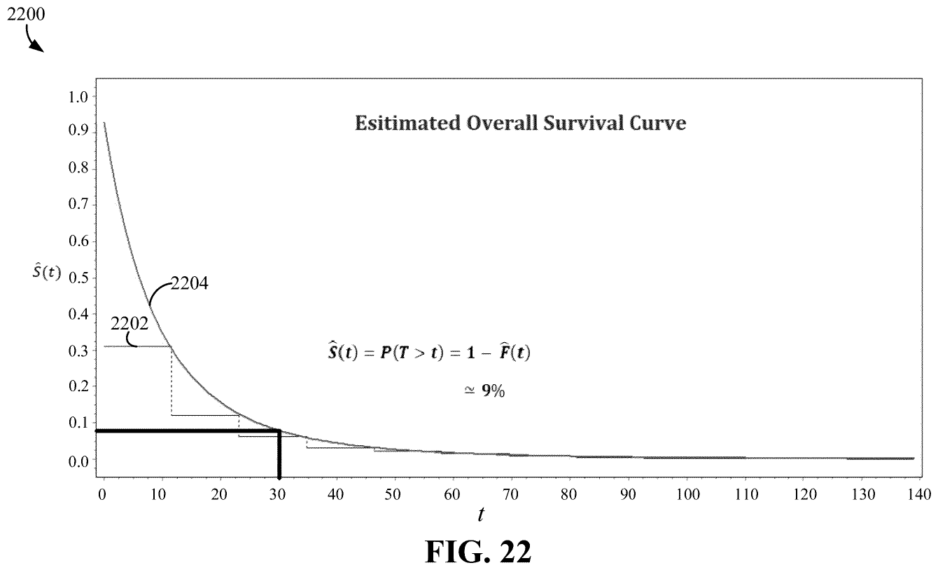

The survival function Ŝ(·;·) can be used to estimate the probability that a randomly selected patient diagnosed with pancreatic cancer would survive beyond time t, which is denoted by P (T≥t). For example, the probability that a patient diagnosed with pancreatic cancer would survive beyond 30 months can be calculated. That is, for t=30 in Equation (3-23), the probability can be estimated as 0.09. Thus, it can be inferred that a randomly chosen pancreatic cancer patient has a 9% chance of survival beyond 30 months. FIG. 22 describes the parametric survival plot for pancreatic cancer patients, generated using GP probability distribution.

Comparison between the Parametric & Nonparametric Survival Estimates: In the parametric analysis above, it is found that patients' survival times (both male and female) with pancreatic cancer follows a Generalized Pareto (GP) distribution and estimated the survival function based on that. The survival function of the two methods can estimate the survival probability of a patient diagnosed with pancreatic cancer beyond a given time. The survival probabilities corresponding to a specific time (in months) are shown in Table 26 for comparison purposes. In Table 26, the probability estimates computed by the GP survival function are significantly higher than the classical non-parametric probability estimates. Since parametric methods are more powerful, robust, and efficient than non-parametric methods, the parametric estimates of the probabilities can be used as the most accurate estimates. In Table 26, Ŝₚ(t) is the parametric survival probability estimates for pancreatic cancer patients using GP probability distribution. Ŝ_{NP}(t) is the nonparametric survival probability estimates for pancreatic cancer patients.

TABLE 26
Table of Comparison of Estimated Survival Probabilities
of Pancreatic Cancer Patients Computed Using Parametric
and Non-Parametric Procedures.

| t | Ŝ_{P}(t) | Ŝ_{NP}(t) |
|---|---|---|
| 0 | .96 | .88 |
| 1 | .87 | .77 |
| 2 | .81 | .69 |
| 3 | .77 | .62 |
| 4 | .7 | .57 |
| 5 | .63 | .52 |
| 6 | .57 | .47 |
| 7 | .51 | .44 |
| 8 | .47 | .4 |
| 9 | .43 | .36 |
| 10 | .39 | .33 |

Results: Given the risk posed by pancreatic cancer in the past several years, it is desirable to investigate the prognosis and enhance the therapeutic/treatment strategy of pancreatic cancer. The primary treatment for most types of pancreatic cancer is chemotherapy, sometimes, along with a targeted therapy drug. A stem cell transplant might follow this. Surgery and radiation therapy do not fall under crucial treatments for pancreatic cancer, but they might be used in exceptional circumstances. Also, the treatment approach for children with pancreatic cancer can be slightly different from that used for adults. Different research approaches and methodologies have been developed to treat pancreatic cancer patients to boost their survival times. In some examples, a data-driven research can be performed on Acute Myeloid Leukemia (AML) by doing some parametric and non-parametric analysis to improve the survival probabilities of patients of different gender groups. In the present disclosure, the inventors analyzed a total of 10,000 patient information and have shown that there was no significant difference between the overall survival times of male and female pancreatic cancer patients. The inventors identified a well-defined probability distribution that characterizes the survival times of a total of 10,000 patient (5,100 male and 4,900 female) diagnosed with pancreatic cancer and used the information to estimate the parametric survival function driven by generalized Pareto (GP) probability distribution. The inventors have tested if there is any significant difference between the mean survival times of male and female patients in each of the four stages. The inventors have identified the probability distributions of male and female survival times in four different cancer stages, and derived their analytical forms. Also we derived the parametric survival functions in each stages, driven by different parametric probability distributions. The inventors calculated the overall survival probabilities utilizing the frequently used classical non-parametric cancer survivorship analysis method and compared those estimates with the parametric probability estimates obtained from GP probability distribution.

Conclusion: In the present disclosure, a new example approach of performing survival analysis is developed by estimating survival probability estimates of patients diagnosed with pancreatic cancer, which is driven by the Generalized Pareto (GP) distribution. The new example parametric method has found to provide the higher estimates of the survival probabilities than the classical non-parametric estimates. The parametric survival analysis's difficulty is the fundamental inherent assumption that the survival times follow a specific probability distribution. But if such restriction can be overcome, more robust and efficient results can be obtained from the parametric analysis, which has greater statistical power. In some examples, the hazard function, which determines the rate at which patients die with pancreatic cancer, after finding the most appropriate parametric distribution can be determined. Depending on the parametric approach utilized for estimating the probability of survival of patients diagnosed with pancreatic cancer, the following important recommendations are imparted.

Given the information regarding male and female cancer patients' survival times, it is customary to investigate first if there exists any statistically significant difference between the male and female patients' true median survival times. If the difference is significant, a separate analysis can be performed for each of the two groups. In the present study, the inventors found that there is no significant difference between overall survival times of male and female patients diagnosed with pancreatic cancer.

After identifying the appropriate probability distributions of male and female cancer patients, if further data is available regarding the different stages, it is desirable to identify the analytical forms of the probability distributions that drive the survival data in each of the four individual stages.

If information is available, then the stage by stage analysis reflects the survival probability of patients in individual stages.

If the only information provided about the patient is the survival time, then estimating the survival probability using the parametric technique will yield more accurate, robust, and efficient results than the classical approaches used.

This disclosure can provide a more effective and plausible method for estimating the survival probability and analysis of cancer survivorship data to further enhance the therapeutic/treatment process of pancreatic cancer.

In some examples, the cancer disease probability distribution function-based model can be used for a patient to estimate a survival chance beyond a time input (e.g., using a medical website portal, or any suitable medical system). For example, as described above, various apps or software portals can be provided to give information to patients and their families, which may include survival times and predictions, and what the families should expect for the patient's health during the remaining predicted survival time. In addition, healthcare/pharmaceutical companies, healthcare professionals/doctors, health science researchers, cancer institutes and departments of public health, health policy makers, biomedical research institutes, government health organizations and/or any other suitable organization can use the cancer disease probability distribution function-based model for medical records, commercial products, research, or any other suitable purposes, such as described above.

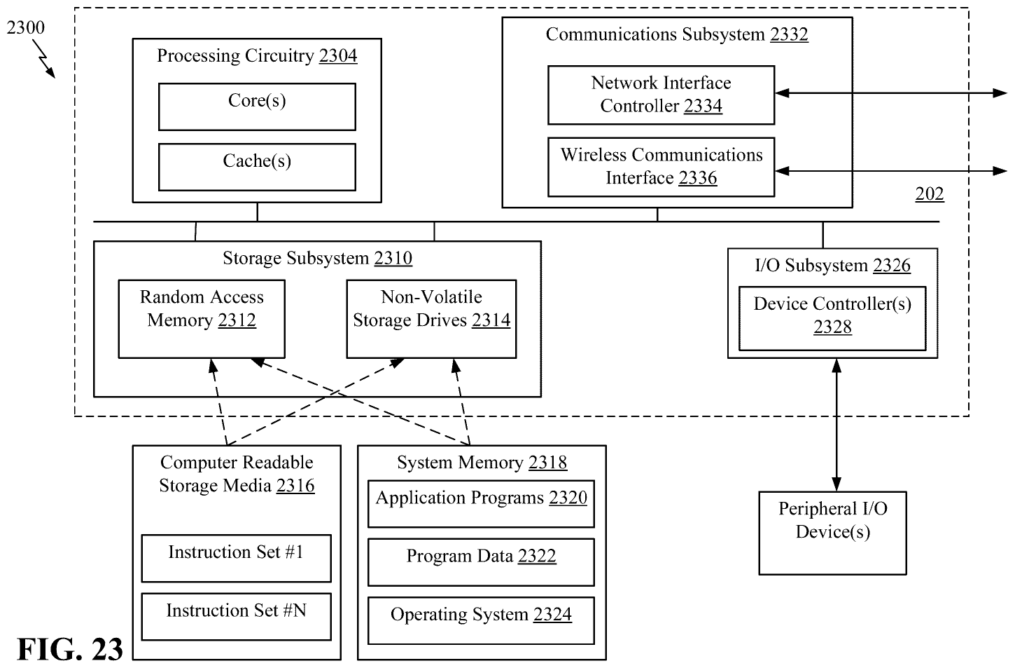

With reference now to FIG. 23, a block diagram of an example computing system 2300 is shown. The computing system 2300 (e.g., one or more computers) may represent an example of one or more server(s), one or more client device(s), or any suitable computing device described herein. In some examples, the computing system 2300 may represent a combination of one or more computing devices and/or servers of a computing environment.

In some examples, the computing system 2300 may include processing circuitry 2304, such as one or more processing unit(s), processor(s), etc. In some examples, the processing circuitry 2304 may communicate (e.g., interface) with a number of peripheral subsystems via a bus subsystem 2302. These peripheral subsystems may include, for example, a storage subsystem 2310, an input/output (I/O) subsystem 2326, and a communications subsystem 2332.

In some examples, the processing circuitry 2304 may be implemented as one or more integrated circuits (e.g., a conventional micro-processor or microcontroller). In an example, the processing circuitry 2304 may control the operation of the computing system 2300. The processing circuitry 2304 may include single core and/or multicore (e.g., quad core, hexa-core, octo-core, ten-core, etc.) processors and processor caches. The processing circuitry 2304 may execute a variety of resident software processes embodied in program code, and may maintain multiple concurrently executing programs or processes. In some examples, the processing circuitry 2304 may include one or more specialized processors, (e.g., digital signal processors (DSPs), outboard, graphics application-specific, and/or other processors).

In some examples, the bus subsystem 2302 provides a mechanism for intended communication between the various components and subsystems of computing system 2300. Although the bus subsystem 2302 is shown schematically as a single bus, alternative embodiments of the bus subsystem may utilize multiple buses. In some examples, the bus subsystem 2302 may include a memory bus, memory controller, peripheral bus, and/or local bus using any of a variety of bus architectures (e.g., Industry Standard Architecture (ISA), Micro Channel Architecture (MCA), Enhanced ISA (EISA), Video Electronics Standards Association (VESA), and/or Peripheral Component Interconnect (PCI) bus, possibly implemented as a Mezzanine bus manufactured to the IEEE P1386.1 standard).

In some examples, the I/O subsystem 2326 may include one or more device controller(s) 2328 for one or more user interface input devices and/or user interface output devices, possibly integrated with the computing system 2300 (e.g., integrated audio/video systems, and/or touchscreen displays), or may be separate peripheral devices which are attachable/detachable from the computing system 2300. Input may include keyboard or mouse input, audio input (e.g., spoken commands), motion sensing, gesture recognition (e.g., eye gestures), etc. As non-limiting examples, input devices may include a keyboard, pointing devices (e.g., mouse, trackball, and associated input), touchpads, touch screens, scroll wheels, click wheels, dials, buttons, switches, keypad, audio input devices, voice command recognition systems, microphones, three dimensional (3D) mice, joysticks, pointing sticks, gamepads, graphic tablets, speakers, digital cameras, digital camcorders, portable media players, webcams, image scanners, fingerprint scanners, barcode readers, 3D scanners, 3D printers, eye gaze tracking devices, and the like. In general, use of the term “output device” is intended to include all possible types of devices and mechanisms for outputting information from computing system 2300, such as to a user (e.g., via a display device) or any other computing system, such as a second computing system 2300. In an example, output devices may include one or more display subsystems and/or display devices that visually convey text, graphics and audio/video information (e.g., cathode ray tube (CRT) displays, flat-panel devices, liquid crystal display (LCD) or plasma display devices, projection devices, touch screens, etc.), and/or may include one or more non-visual display subsystems and/or non-visual display devices, such as audio output devices, etc. As non-limiting examples, output devices may include, indicator lights, monitors, printers, speakers, headphones, automotive navigation systems, plotters, voice output devices, modems, etc.

In some examples, the computing system 2300 may include one or more storage subsystems 2310, including hardware and software components used for storing data and program instructions, such as system memory 2318 and computer-readable storage media 2316. In some examples, the system memory 2318 and/or the computer-readable storage media 2316 may store and/or include program instructions that are loadable and executable on the processor(s) 2304. In an example, the system memory 2318 may load and/or execute an operating system 2324, program data 2322, server applications, application program(s) 2320 (e.g., client applications), Internet browsers, mid-tier applications, etc. In some examples, the system memory 2318 may further store data generated during execution of these instructions.

In some examples, the system memory 2318 may be stored in volatile memory (e.g., random-access memory (RAM) 2312, including static random-access memory (SRAM) or dynamic random-access memory (DRAM)). In an example, the RAM 2312 may contain data and/or program modules that are immediately accessible to and/or operated and executed by the processing circuitry 2304. In some examples, the system memory 2318 may also be stored in non-volatile storage drives 2314 (e.g., read-only memory (ROM), flash memory, etc.). In an example, a basic input/output system (BIOS), containing the basic routines that help to transfer information between elements within the computing system 2300 (e.g., during start-up), may typically be stored in the non-volatile storage drives 2314.

In some examples, the storage subsystem 2310 may include one or more tangible computer-readable storage media 2316 for storing the basic programming and data constructs that provide the functionality of some embodiments. In an example, the storage subsystem 2310 may include software, programs, code modules, instructions, etc., that may be executed by the processing circuitry 2304, in order to provide the functionality described herein. In some examples, data generated from the executed software, programs, code, modules, or instructions may be stored within a data storage repository within the storage subsystem 2310. In some examples, the storage subsystem 2310 may also include a computer-readable storage media reader connected to the computer-readable storage media 2316.

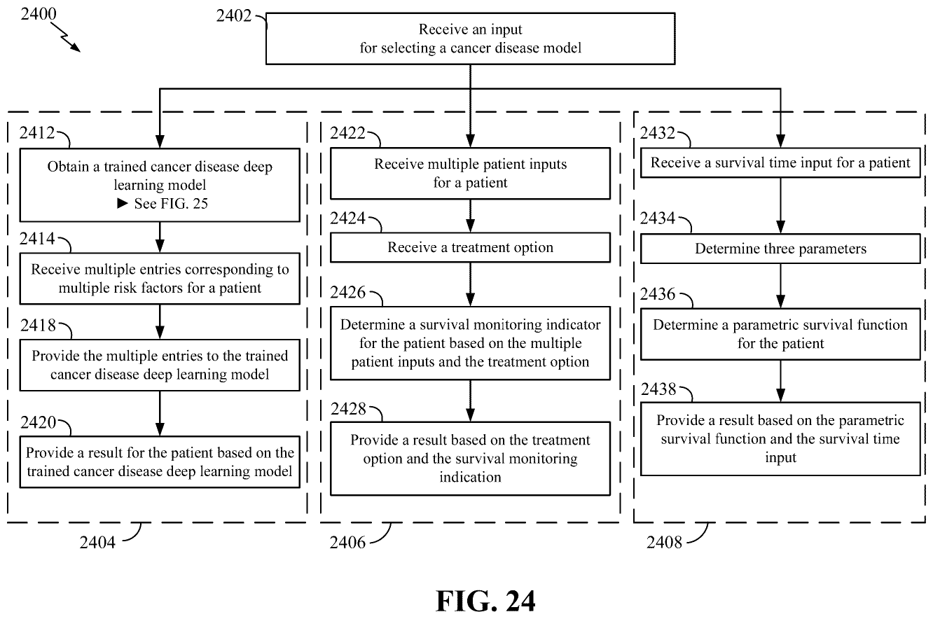

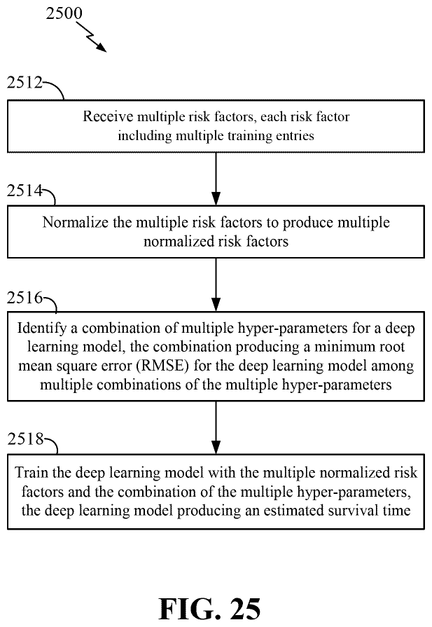

In some examples, the computer-readable storage media 2316 may contain program code, or portions of program code. Together and optionally, in combination with the system memory 2318, the computer-readable storage media 2316 may comprehensively represent remote, local, fixed, and/or removable storage devices plus storage media for temporarily and/or more permanently containing, storing, transmitting, and/or retrieving computer-readable information. In some examples, the computer-readable storage media 2316 may include any appropriate media known or used in the art, including storage media and communication media, such as but not limited to, volatile and non-volatile, removable and non-removable media implemented in any method or technology for storage and/or transmission of information. In some examples, the computer-readable storage media 2316 can include a trained disease deep learning mode, a cancer disease stochastic model, a cancer disease probability distribution model, and instructions to perform process 2400 in FIG. 24. In further examples, the computer-readable storage media 2316 can further include instructions to perform process 2500 in FIG. 25. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer-readable media. This can also include nontangible computer-readable media, such as data signals, data transmissions, or any other medium which can be used to transmit the desired information and which can be accessed by the computing system 2300. In an illustrative and non-limiting example, the computer-readable storage media 2316 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media.

In some examples, the computer-readable storage media 2316 may include, but is not limited to, Zip® drives, flash memory cards, universal serial bus (USB) flash drives, secure digital (SD) cards, DVD disks, digital video tape, and the like. In some examples, the computer-readable storage media 2316 may include, solid-state drives (SSD) based on non-volatile memory such as flash-memory based SSDs, enterprise flash drives, solid state ROM, and the like, SSDs based on volatile memory such as solid-state RAM, dynamic RAM, static RAM, DRAM-based SSDs, magneto-resistive RAM (MRAM) SSDs, and hybrid SSDs that use a combination of DRAM and flash memory-based SSDs. The disk drives and their associated computer-readable media may provide non-volatile storage of computer-readable instructions, data structures, program modules, and other data for the computing system 2300.

In some examples, the communications subsystem 2332 may provide a communication interface from the computing system 2300 and external computing devices via one or more communication networks, including local area networks (LANs), wide area networks (WANs) (e.g., the Internet), and various wireless telecommunications networks. As illustrated in FIG. 23, the communications subsystem 2332 may include, for example, one or more network interface controllers (NICs) 2334, such as Ethernet cards, Asynchronous Transfer Mode NICs, Token Ring NICs, and the like, as well as one or more wireless communications interfaces 2336, such as wireless network interface controllers (WNICs), wireless network adapters, and the like. Additionally, and/or alternatively, the communications subsystem 2332 may include one or more modems (telephone, satellite, cable, ISDN), synchronous or asynchronous digital subscriber line (DSL) units, Fire Wire® interfaces, USB® interfaces, and the like. Communications subsystem 2332 also may include radio frequency (RF) transceiver components for accessing wireless voice and/or data networks (e.g., using cellular telephone technology, advanced data network technology, such as 3G, 4G, 5G, or EDGE (enhanced data rates for global evolution), Wi-Fi (IEEE 802.11 family standards, or other mobile communication technologies, or any combination thereof), global positioning system (GPS) receiver components, and/or other components.

In some examples, the communications subsystem 2332 may also receive input communication in the form of structured and/or unstructured data feeds, event streams, event updates, and the like, on behalf of one or more users who may use or access the computing system 2300. In an example, the communications subsystem 2332 may be configured to receive an input to select a cancer disease model, patient inputs data, or any user input. Additionally, the communications subsystem 2332 may be configured to receive data in the form of continuous data streams, which may include event streams of real-time events and/or event updates (e.g., sensor data applications, financial tickers, network performance measuring tools, clickstream analysis tools, automobile traffic monitoring, etc.). In some examples, the communications subsystem 2332 may output such structured and/or unstructured data feeds (e.g., a result of a cancer disease model), event streams, event updates, and the like to one or more data stores that may be in communication with one or more streaming data source computing systems (e.g., one or more data source computers, etc.) coupled to the computing system 2300. The various physical components of the communications subsystem 2332 may be detachable components coupled to the computing system 2300 via a computer network (e.g., a communication network 120), a FireWire® bus, or the like, and/or may be physically integrated onto a motherboard of the computing system 2300. In some examples, the communications subsystem 2332 may be implemented in whole or in part by software.

Due to the ever-changing nature of computers and networks, the description of the computing system 2300 depicted in the figure is intended only as a specific example. Many other configurations having more or fewer components than the system depicted in the figure are possible. For example, customized hardware might also be used and/or particular elements might be implemented in hardware, firmware, software, or a combination. Further, connection to other computing devices, such as network input/output devices, may be employed. Based on the disclosure and teachings provided herein, a person of ordinary skill in the art will appreciate other ways and/or methods to implement the various embodiments.

FIG. 24 is a flow diagram illustrating an example process 2400 for cancer disease estimation in accordance with some aspects of the present disclosure. As described below, a particular implementation can omit some or all illustrated features/steps, may be implemented in some embodiments in a different order, and may not require some illustrated features to implement all embodiments. In some examples, the computing device 2310 in connection with FIG. 23 can be used to perform the example process 2400. However, it should be appreciated that any suitable apparatus or means for carrying out the operations or features described below may perform the process 2400.

At step 2402, process 2400 can receive an input for selecting a cancer disease prediction model. In some examples, step 2402 can be implemented on a web browser, which connected to a computing device 2310 via the communication network, or a display on the computing device 2310. For example, a user can select a cancer disease prediction model (e.g., using a check box, radio button, toggle-switch, choice chips, multi-select chips, etc.) among three cancer disease models (i.e., a cancer disease deep learning model 2404 (i.e., steps 2412-2420), a cancer disease stochastic model 2406 (i.e., steps 2422-2428), or a probability distribution function-based model 2408 (i.e., steps 2432-2438)) based on the input. In some examples, the input can be a user input (e.g., a click or selection of the analytical model) or a non-human input (e.g., an automatic selection of the analytical model based on a location of the user, a user profile, and/or any other suitable parameters). In further examples, the received input can include at least one of: a first user request for information relating to an estimated survival time, a second user request for information relating to a changing survival time due to a treatment option, or a third user request for information relating to a survival chance beyond a time input. The first user request causes a first selection of the cancer disease deep learning model. The second user request causes a second selection of the cancer disease stochastic model. The third user request causes a third selection of the cancer disease probability distribution function-based model. In response to the input, the process 2400 can perform the selected process (e.g., using a cancer disease deep learning model 2404 at steps 2412-2420, using a cancer disease stochastic model 2406 at steps 2422-2428, using a probability distribution function-based model 2408 at steps 2432-2438, or using any other suitable cancer disease model).

Steps 2412-2420 are an example process for a cancer disease deep learning model 2404. At step 2412, process 2400 can obtain a trained deep learning model. As a non-limiting example, process 2500 in FIG. 25 illustrates an example process for cancer disease model training.

At step 2414, process 2400 can receive multiple entries corresponding multiple risk factors for a patient. For example, the multiple risk factors can include, but are not limited to: an age, a cancer stage, an indication of regular aspirin usage, an indication of regular Ibuprofen usage, a number of first-degree relatives with any type of cancer, a sex of the patient, a current body mass index at baseline, a total number of smoking years, an indication of the existence history of gall bladder stones or inflammation, and/or an indication of blood pressure history. However, it should be appreciated that the risk factors are not limited to the recited list. In some examples, the risk factors can be reduced to less than listed factors (e.g., most contributing risk factors (e.g., age, current, BMI, the number of years the patient smoked cigarette, family history of cancer, and/or aspirin regular usage)). In other examples, the risk factors can further include any other suitable factors. As a non-limiting example, the multiple entries corresponding to multiple risk factors can include an age of the patient, a cancer stage of the patient, an indication of regular aspirin usage, an indication of regular Ibuprofen usage, a number of first-degree relatives of the patient with any type of cancer, a sex of the patient, a current body mass index of the patient at baseline, a total number of years the patient smoked, an indication of whether the patient has ever gall bladder stones or inflammation, and/or an indication of whether the patient has ever high blood pressure.

In some examples, a risk factor can include a categorical entry among the multiple entries. For example, some risk factors (e.g., the cancer stage of the patient (e.g., a) localized, b) regional, and c) distant), an indication of regular aspirin usage (e.g., yes or no), an indication of regular Ibuprofen usage (e.g., yes or no), a sex of the patient (e.g., male or female), an indication of whether the patient has ever gall bladder stones or inflammation (e.g., yes or no), and an indication of whether the patient has ever high blood pressure (e.g., yes or no)) can be categorical. On the other hand, other risk factors (e.g., an age of the patient, a number of first-degree relatives of the patient with any type of cancer, a current body mass index of the patient at baseline, and a total number of years the patient smoked) can be numeric. In further examples, process 2400 can convert categorical entries of the multiple entries to numeric entries for the cancer disease deep learning model to process numeric entries. In some examples, the process 2400 can further update the categorical risk factors based on the numeric values. For example, a categorical entry of each categorical risk factor can be converted to a numerical form (e.g., using one-hot-encoding).

At step 2418, process 2400 can provide the multiple entries for the patient including converted numerical entries to the trained cancer disease deep learning model. In some examples, process 2400 can receive a probability of an estimated survival time of the patient from the trained cancer disease deep learning model. In some examples, the trained cancer disease deep learning model can include a boosted regression tree model. As a non-limiting example, the trained cancer disease deep learning model can have a sum of 158±20 decision trees (e.g., between 138 decision trees and 178 decision trees) with a combination of a plurality of identified hyper-parameters. As a non-limiting example, the trained cancer disease deep learning model can use three hyperbolic tangent (tanh) activation functions in three corresponding hidden layers of the deep learning model. As a non-limiting example, the trained cancer disease deep learning model can use a dropout, a batch-normalization, and an Adam optimizer. The cancer disease deep learning model is further described in connection with process 2500 in FIG. 25.

At step 2420, process 2400 can provide a result for the patient based on the trained cancer disease deep learning model. For example, process 2400 can display the result (e.g., the probability of an estimated survival time).

Steps 2522-2428 are an example process for a cancer disease stochastic model 2406. At step 2422, process 2400 can receive multiple patient inputs for a patient. In some examples, the multiple patient inputs can include: a race indication indicative of a race of the patient, an age group indication indicative of an age group of the patient, and a cancer stage indication indicative of a cancer stage of the patient. As a non-limiting scenario, the age group is one of a first age group between age 40 to age 59, a second age group between age 60 to age 79, and a third age group between age 80 and above. In some examples, the age group indication can include a number, a character, a symbol, or any other suitable indication to indicate the first, second, and third groups. As a non-limiting scenario, the race is one of Caucasian, African-American, and others. In some examples, the race indication can include a number, a character, a symbol, or any other suitable indication to indicate three races (i.e., Caucasian, African-American, and others). As a non-limiting scenario, the cancer stage is one of stage 1, stage 2, stage 3, and stage 4. In some examples, the cancer stage indication can include a number, a character, a symbol, or any other suitable indication to indicate four stages (i.e., stage 1, stage 2, stage 3, and stage 4). In some examples, a user can provide the multiple patient inputs on a webpage, a display, or any suitable means, and process 2400 can receive the multiple patient inputs (e.g., from the webpage, the display or any suitable means). In further examples, the multiple patient inputs can further include a gender indication indicative of a gender of the patient.

At step 2424, process 2400 can receive a treatment option. In some examples, the treatment option can be one of a first indication (e.g., C) indicative of chemotherapy, a second indication (e.g., R) indicative of radiation, and a third indication (e.g., C+R) indicative of a combination of chemotherapy and radiation. In further examples, each of the first, second, and third indications can include a number, a character, a symbol, or any other suitable indication to indicate chemotherapy, radiation, or the combination of chemotherapy and radiation. It should be appreciated that the treatment option is not limited to the listed treatment options. For example, the treatment option can be a surgery, immunotherapy, a targeted therapy, or any other suitable treatment option. In further examples, process 2400 can receive multiple treatment options to compare results of different treatment options.

At step 2426, process 2400 can determine a survival monitoring indicator (SMI) for the patient based on the multiple patient inputs and the treatment option. In some examples, the SMI for the patient can be determined by:

$${SMI}_{j k}^{l m} = \frac{{n}_{j k}^{l m}}{{\sum }_{i = 1}^{n} log ( \frac{{( {t}_{n} )}_{j k}^{l m}}{{( {t}_{i} )}_{j k}^{l m}} )} ,$$

where j is the treatment option, k is the cancer stage indicator indicative, l is the age group indicator, m is the race indicator, SMIⱼₖ^{lm} is the survival monitoring indicator of the treatment (j) at the cancer stage (k) for the group age (l) belonging to the race (m), (tᵢ)ⱼₖ^{lm} is a longest time to death, and nⱼₖ^{lm} is a number of patients.

At step 2428, process 2400 can provide a result based on the treatment option and the survival monitoring indication. In some examples, the result can indicate an increasing survival time of the patient, a decreasing survival time of the patient, or a same survival time of the patient based on the treatment option. Based on the result, a user can appreciate whether the treatment option is effective or not.

In further examples, process 2400 can further determine a survival intensity function (SIF) based on the SMI. As a non-limiting example, in response to the survival intensity function decreasing with time, the result can indicate the increasing survival time of the patient. As a non-limiting example, in response to the survival intensity function increasing with the time, the result can indicate the decreasing survival time of the patient. As a non-limiting example, in response to the survival intensity function being constant with time, the result can indicate the same survival time. In even further examples, process 2400 can further display a survival rate of changing of the patient based on the survival intensity function.

At step 2432, process 2400 can receive a survival time input for a patient. In some example, the survival time input can include a future time instant (e.g., 3 months, 5 months, 10 months, 30 months, or any suitable further time point). In further examples, a user can provide the survival time input via a website, a display, or any suitable device to provide the survival time input to a computing device to perform process 2400.

At step 2434, process 2400 can determine three parameters. For example, the three parameters can include a location parameter (\hat{k}), a scale parameter (\hat{δ}), and a shape parameter (\hat{μ}). As a non-limiting example, the location parameter is determined by:

$$\hat{k} = \frac{{α}_{0} - 8 {α}_{1} - 9 {α}_{2}}{- {α}_{0} + {α}_{1} - {α}_{2}} ,$$

the scale parameter is determined by:

$$\hat{σ} = \frac{( {α}_{0} - 2 {α}_{1} ) ( {α}_{0} - 3 {α}_{2} ) ( - 4 {α}_{1} + 6 {α}_{2} )}{{( - {α}_{0} + 4 {α}_{1} - 3 {α}_{2} )}^{2}} ,$$

and the shape parameter is determined by:

$$\hat{μ} = \frac{2 {α}_{0} {α}_{1} - 6 {α}_{0} {α}_{2} + 6 {α}_{1} {α}_{2}}{- {α}_{0} + 4 {α}_{1} - 3 {α}_{2}} .$$

In some scenarios,

$${α}_{0} = ( μ + \frac{σ}{k} ) - \frac{σ}{k} ( \frac{1}{k + 1} ) ,$$

$${α}_{1} = \frac{1}{2} ( μ + \frac{σ}{k} ) - \frac{σ}{k} ( \frac{1}{k + 2} ) , and$$

$${α}_{2} = \frac{1}{3} ( μ + \frac{σ}{k} ) - \frac{σ}{k} ( \frac{1}{k + 3} ) .$$

At step 2436, process 2400 can determine a parametric survival function for the patient. In some examples, the parametric survival function can be determined by:

$$\hat{S} ( the parametric survival function ) = [ 1 + the shape parameter {( \frac{the survival time input - the location parameter}{the scale parameter} )}^{- 4.54} ] .$$

In further examples, inventors can estimate the location parameter, the scale parameter, and the shape parameter as 0.65, 8.9, and 0.22, respectively. Then, the parametric survival function can be:

$$\hat{S} = [ 1 + 0.22 {( \frac{the survival time input - 0.65}{8.9} )}^{- 4.54} ] .$$

At step 2438, process 2400 can provide a result based on the parametric survival function and the survival time input. In some examples, the result can be a chance of survival beyond the survival time input. In the examples above, when the survival time input is 30 months, the probability output of the parametric survival function is

$$0.09 \approx [ 1 + 0.22 {( \frac{30 - 0.65}{8.9} )}^{- 4.54} ] .$$

Thus, the result can be a 9% chance of survival beyond 30 months.

In some examples, process 2400 can be used for healthcare/pharmaceutical companies, healthcare professionals/doctors, health science researchers, cancer institutes and departments of public health, health policy makers, biomedical research institutes, government health organizations and/or any other suitable organization.

FIG. 25 is a flow diagram illustrating an example process 2500 for cancer disease deep learning model training in accordance with some aspects of the present disclosure. As described below, a particular implementation can omit some or all illustrated features/steps, may be implemented in some embodiments in a different order, and may not require some illustrated features to implement all embodiments. In some examples, the computing system 2300 in connection with FIG. 23 can be used to perform the example process 2500. However, it should be appreciated that any suitable apparatus or means for carrying out the operations or features described below may perform the process 2500.

At step 2512, process 2500 can receive multiple risk factors. Each risk factor of the multiple risk factors can include multiple training entries. In a non-limiting scenario, a patient can have multiple training entries corresponding to the multiple risk factors. For example, the multiple risk factors can include, but are not limited to: an age of the patient, a cancer stage of the patient, an indication of regular aspirin usage, an indication of regular Ibuprofen usage, a number of first-degree relatives of the patient with any type of cancer, a sex of the patient, a current body mass index of the patient at baseline, a total number of years the patient smoked, an indication of whether the patient has ever gall bladder stones or inflammation, and/or an indication of whether the patient has ever high blood pressure. However, it should be appreciated that the risk factors are not limited to the recited list. In some examples, the risk factors can be reduced to less than listed factors (e.g., most contributing risk factors (e.g., age, current, BMI, the number of years the patient smoked cigarette, family history of cancer, and/or aspirin regular usage)). In other examples, the risk factors can further include any other suitable factors. In further examples, process 2500 can receive multiple patients' risk factor information for training and developing a deep learning model. Thus, multiple training entries corresponding to multiple risk factors for a patient can be a set of patient information, and process 2500 can receive multiple sets of multiple training entries corresponding to multiple patients.

In further examples, a risk factor can include a categorical entry, and multiple categorical entries for the risk factor can correspond to the multiple training entries associated with the risk factor. For example, some risk factors (e.g., the cancer stage of the patient (e.g., a) localized, b) regional, and c) distant), an indication of regular aspirin usage (e.g., yes or no), an indication of regular Ibuprofen usage (e.g., yes or no), a sex of the patient (e.g., male or female), an indication of whether the patient has ever gall bladder stones or inflammation (e.g., yes or no), and an indication of whether the patient has ever high blood pressure (e.g., yes or no)) can be categorical. On the other hand, other risk factors (e.g., an age of the patient, a number of first-degree relatives of the patient with any type of cancer, a current body mass index of the patient at baseline, and a total number of years the patient smoked) can be numeric. Thus, a categorical risk factor can include multiple categorical entries corresponding multiple patients while a numeric risk factor can include multiple numeric entries corresponding multiple patients.

In further examples, process 2500 can further convert the multiple categorical entries to multiple numeric values corresponding to the multiple categorical entries. Then, the process 2500 can further update the risk factor based on the multiple numeric values. For example, a categorical entry of each categorical risk factor can be converted to a numerical form (e.g., using one-hot-encoding).

At step 2514, process 2500 can normalize the multiple risk factors to produce multiple normalized risk factors. For example, the multiple risk factors can be normalized using Min-Max normalization. The Min-Max normalization can be expresses as:

$$y *= \frac{y - min ( y )}{max ( y ) - min ( y )} ,$$

where y* is a normalized entry of a normalized risk factor of the multiple normalized risk factors, y is a training entry of the multiple training entries in a risk factor of the multiple risk factors, max(y) is a maximum value among the multiple entries in the risk factor, and min(y) is a minimum value among the multiple training entries in the risk factor.

At step 2516, process 2500 can identify a combination of multiple hyper-parameters for a deep learning model. In some examples, the combination can produce a minimum root mean square error (RMSE) for the deep learning model among multiple combinations of the multiple identified hyper-parameters. In further examples, the multiple hyper-parameters can include at least one of: an estimated time to train the deep learning model, a maximum depth of a tree of the deep learning model, a smallest number of instances in a child node of the deep learning model, a subsample ratio for regulating a number of samples provided to the tree of the deep learning model, or a column ratio when constructing the tree of the deep learning model. For example, the estimated time is set as 0.05±0.01 (i.e., 0.04≤estimated time≤0.06), the maximum depth is set as 7±1 (i.e., 6≤maximum depth≤8), the smallest number of instances is set as 1±1 (i.e. 0≤smallest number of instances≤2), the subsample ratio is set as 0.8±0.1 (i.e., 0.7≤subsample ratio≤0.9), and the column ratio is set as 0.8±0.1 (i.e., 0.7≤column ration≤0.9).

At step 2518, process 2500 can train the deep learning model with the multiple normalized risk factors and the combination of the multiple identified hyper-parameters. In some examples, the deep learning model can produce an estimated survival time of a patient. In further examples, the deep learning model can include a boosted regression tree model. In even further examples, the deep learning model is a sum of 158±20 decision trees with the combination of the multiple identified hyper-parameters. In further examples, the deep learning model can use three hyperbolic tangent (tanh) activation functions in three corresponding hidden layers of the deep learning model. In further examples, the deep learning model uses a dropout, a batch-normalization, and an Adam optimizer. In further examples, the multiple risk factors can have different contributing weights for the deep learning model to process.

### Further Examples Having a Variety of Features:

Example 1: A method, apparatus, and non-transitory computer-readable medium for cancer disease estimation, comprising: receiving an input for selecting at least one of: a cancer disease deep learning model, a cancer disease stochastic model, or a cancer disease probability distribution function-based model; in response to the input to select the cancer disease deep learning model, obtaining a trained cancer disease deep learning model; receiving a plurality of entries corresponding to a plurality of risk factors for a patient; providing the plurality of entries to the trained cancer disease deep learning model; and providing a first result for the patient based on the trained cancer disease deep learning model; in response to the input to select the cancer disease stochastic model, receiving a plurality of patient inputs for the patient; receiving a treatment option; determining a survival monitoring indicator for the patient based on the plurality of patient inputs and the treatment option; and producing a second result based on the treatment option and the survival monitoring indication; and in response to the input to select the cancer disease probability distribution function-based model, receiving a survival time input for the patient; determining three parameters; determining a parametric survival function for the patient; and providing a third result based on the parametric survival function and the survival time input.
Example 2: A method, apparatus, and non-transitory computer-readable medium of Example 1, wherein the received input comprises at least one of: a first user request for information relating to an estimated survival time, a second user request for information relating to a changing survival time due to a treatment option, or a third user request for information relating to a survival chance beyond a time input, wherein the first user request causes a first selection of the cancer disease deep learning model, wherein the second user request causes a second selection of the cancer disease stochastic model, and wherein the third user request causes a third selection of the cancer disease probability distribution function-based model.
Example 3: A method, apparatus, and non-transitory computer-readable medium of any of Examples 1 to 2, wherein a risk factor of the plurality of risk factors comprises a categorical entry among the plurality of entries, and wherein the method further comprises: in response to the input to select the cancer disease deep learning model, converting the categorical entry to a numeric value; and updating an entry corresponding to the categorical entry based on the of numeric value.
Example 4: A method, apparatus, and non-transitory computer-readable medium of any of Examples 1 to 3, wherein the plurality of entries corresponding to the plurality of risk factors comprises: an age of the patient, a cancer stage of the patient, an indication of regular aspirin usage, an indication of regular Ibuprofen usage, a number of first-degree relatives of the patient with any type of cancer, a sex of the patient, a current body mass index of the patient at baseline, a total number of years the patient smoked, an indication of whether the patient has ever gall bladder stones or inflammation, and an indication of whether the patient has ever high blood pressure.
Example 5: A method, apparatus, and non-transitory computer-readable medium of any of Examples 1 to 4, wherein the trained cancer disease deep learning model comprises a boosted regression tree model.
Example 6: A method, apparatus, and non-transitory computer-readable medium of any of Examples 1 to 5, wherein the trained cancer disease deep learning model is a sum of 158±20 decision trees with a combination of a plurality of identified hyper-parameters.
Example 7: A method, apparatus, and non-transitory computer-readable medium of any of Examples 1 to 6, wherein the trained cancer disease deep learning model uses three hyperbolic tangent (tanh) activation functions in three corresponding hidden layers of the deep learning model.
Example 8: A method, apparatus, and non-transitory computer-readable medium of any of Examples 1 to 7, wherein the trained cancer disease deep learning model uses a dropout, a batch-normalization, and an Adam optimizer.
Example 9: A method, apparatus, and non-transitory computer-readable medium of any of Examples 1 to 8, wherein the plurality of patient inputs comprises: a race indication indicative of a race of the patient, an age group indication indicative of an age group of the patient, and a cancer stage indication indicative of a cancer stage of the patient.
Example 10: A method, apparatus, and non-transitory computer-readable medium of any of Examples 1 to 9, wherein the plurality of patient inputs further comprises a gender indication indicative of a gender of the patient.
Example 11: A method, apparatus, and non-transitory computer-readable medium of any of Examples 1 to 10, wherein the age group is one of a first age group between age 40 to age 59, a second age group between age 60 to age 79, and a third age group between age 80 and above.
Example 12: A method, apparatus, and non-transitory computer-readable medium of any of Examples 1 to 11, wherein the race is one of Caucasian, African-American, and others.
Example 13: A method, apparatus, and non-transitory computer-readable medium of any of Examples 1 to 12, wherein the cancer stage is one of stage 1, stage 2, stage 3, and stage 4.
Example 14: A method, apparatus, and non-transitory computer-readable medium of any of Examples 1 to 13, wherein the survival monitoring indicator for the patient is determined by:

$${SMI}_{jk}^{lm} = \frac{{n}_{jk}^{lm}}{{\sum }_{i = 1}^{n} log ( \frac{{( {t}_{n} )}_{jk}^{lm}}{{( {t}_{i} )}_{jk}^{lm}} )} ,$$

where j is the treatment option, k is the cancer stage indicator indicative, l is the age group indicator, m is the race indicator, SMIⱼₖ^{lm} is the survival monitoring indicator of the treatment (j) at the cancer stage (k) for the group age (l) belonging to the race (m), (tᵢ)ⱼₖ^{lm} is a longest time to death, and nⱼₖ^{lm} is a number of patients.
Example 15: A method, apparatus, and non-transitory computer-readable medium of any of Examples 1 to 14, wherein the treatment option is one of chemotherapy, radiation, and a combination of chemotherapy and radiation.
Example 16: A method, apparatus, and non-transitory computer-readable medium of any of Examples 1 to 15, wherein the result indicates an increasing survival time of the patient, a decreasing survival time of the patient, or a same survival time of the patient.
Example 17: A method, apparatus, and non-transitory computer-readable medium of any of Examples 1 to 16, further comprising: determining a survival intensity function based on the survival monitoring indicator, wherein in response to the survival intensity function decreasing with time, the result indicates the increasing survival time of the patient, wherein in response to the survival intensity function increasing with the time, the result indicates the decreasing survival time of the patient, and wherein in response to the survival intensity function being constant with time, the result indicates the same survival time.
Example 18: A method, apparatus, and non-transitory computer-readable medium of any of Examples 1 to 17, further comprising: displaying a survival rate of changing of the patient based on the survival intensity function.
Example 19: A method, apparatus, and non-transitory computer-readable medium of any of Examples 1 to 18, wherein the three parameters comprise a location parameter (\hat{k}), a scale parameter (\hat{σ}), and a shape parameter (\hat{μ}), wherein the location parameter is determined by:

$$\hat{k} = \frac{{α}_{0} - 8 {α}_{1} - 9 {α}_{2}}{- {α}_{0} + {α}_{1} - {α}_{2}} ,$$

wherein the scale parameter is determined by:

$$\hat{σ} = \frac{( {α}_{0} - 2 {α}_{1} ) ( {α}_{0} - 3 {α}_{2} ) ( - 4 {α}_{1} + 6 {α}_{2} )}{{( - {α}_{0} + 4 {α}_{1} - 3 {α}_{2} )}^{2}} ,$$

wherein the shape parameter is determined by:

$$\hat{μ} = \frac{2 {α}_{0} {α}_{1} - 6 {α}_{0} {α}_{2} + 6 {α}_{1} {α}_{2}}{- {α}_{0} + 4 {α}_{1} - 3 {α}_{2}} , \\ wherein {α}_{0} = ( μ + \frac{σ}{k} ) - \frac{σ}{k} ( \frac{1}{k + 1} ) , \\ wherein {α}_{1} = \frac{1}{2} ( μ + \frac{σ}{k} ) - \frac{σ}{k} ( \frac{1}{k + 2} ) , \\ and wherein {α}_{2} = \frac{1}{3} ( μ + \frac{σ}{k} ) - \frac{σ}{k} ( \frac{1}{k + 3} ) .$$

Example 20: A method, apparatus, and non-transitory computer-readable

medium of any of Examples 1 to 19, wherein the parametric survival function (Ŝ) is determined by:

$$\hat{S} = [ 1 + the shape parameter {( \frac{\begin{matrix}the survival \\ time input - the \\ location parameter\end{matrix}}{the scale parameter} )}^{- 4.54} ] .$$

Example 21: A method, apparatus, and non-transitory computer-readable medium for cancer disease model training, comprising: receiving a plurality of risk factors, each risk factor of the plurality of risk factors comprising a plurality of training entries; normalizing the plurality of risk factors to produce a plurality of normalized risk factors; identifying a combination of a plurality of hyper-parameters for a deep learning model, the combination producing a minimum root mean square error (RMSE) for the deep learning model among a plurality of combinations of the plurality of identified hyper-parameters; and training the deep learning model with the plurality of normalized risk factors and the combination of the plurality of identified hyper-parameters, the deep learning model producing an estimated survival time.
Example 22: A method, apparatus, and non-transitory computer-readable medium of Example 21, wherein a risk factor of the plurality of risk factors comprises a plurality of categorical entries, the plurality of categorical entries corresponding to the plurality of training entries associated with the risk factor, and wherein the method further comprises: converting the plurality of categorical entries to a plurality of numeric values corresponding to the plurality of categorical entries; and updating the risk factor based on the plurality of numeric values.
Example 23: A method, apparatus, and non-transitory computer-readable medium of any of Examples 21 to 22, wherein the plurality of risk factors has different contributing weights.
Example 24: A method, apparatus, and non-transitory computer-readable medium of any of Examples 21 to 23, wherein the plurality of risk factors is normalized using Min-Max normalization being expressed as:

$$y *= \frac{y - min ( y )}{max ( y ) - min ( y )} ,$$

where y* is a normalized entry of a normalized risk factor of the plurality of normalized risk factors, y is a training entry of the plurality of training entries in a risk factor of the plurality of risk factors, max(y) is a maximum value among the plurality of entries in the risk factor, and min(y) is a minimum value among the plurality of training entries in the risk factor.
Example 25: A method, apparatus, and non-transitory computer-readable medium of any of Examples 21 to 24, wherein the plurality of hyper-parameters comprises at least one of: an estimated time to train the deep learning model, a maximum depth of a tree of the deep learning model, a smallest number of instances in a child node of the deep learning model, a subsample ratio for regulating a number of samples provided to the tree of the deep learning model, or a column ratio when constructing the tree of the deep learning model.
Example 26: A method, apparatus, and non-transitory computer-readable medium of any of Examples 21 to 25, wherein the estimated time is set as 0.05±0.01, the maximum depth is set as 7 ±1, the smallest number of instances is set as 1±1, the subsample ratio is set as 0.8±0.1, and the column ratio is set as 0.8±0.1.
Example 27: A method, apparatus, and non-transitory computer-readable medium of any of Examples 21 to 26, wherein the deep learning model comprises a boosted regression tree model.
Example 28: A method, apparatus, and non-transitory computer-readable medium of any of Examples 21 to 27, wherein the deep learning model is a sum of 158±20 decision trees with the combination of the plurality of identified hyper-parameters.
Example 29: A method, apparatus, and non-transitory computer-readable medium of any of Examples 21 to 28, wherein the deep learning model uses three hyperbolic tangent (tanh) activation functions in three corresponding hidden layers of the deep learning model.
Example 30: A method, apparatus, and non-transitory computer-readable medium of any of Examples 21 to 29, wherein the deep learning model uses a dropout, a batch-normalization, and an Adam optimizer.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

What is claimed is:

1. A method for cancer disease estimation, comprising:
receiving an input for selecting at least one of: a cancer disease deep learning model, a cancer disease stochastic model, or a cancer disease probability distribution function-based model;
in response to the input to select the cancer disease deep learning model,
obtaining a trained cancer disease deep learning model;
receiving a plurality of entries corresponding to a plurality of risk factors for a patient;
providing the plurality of entries to the trained cancer disease deep learning model; and
providing a first result for the patient based on the trained cancer disease deep learning model;
in response to the input to select the cancer disease stochastic model, receiving a plurality of patient inputs for the patient;
receiving a treatment option;
determining a survival monitoring indicator for the patient based on the plurality of patient inputs and the treatment option; and
producing a second result based on the treatment option and the survival monitoring indication; and
in response to the input to select the cancer disease probability distribution function-based model,
receiving a survival time input for the patient;
determining three parameters;
determining a parametric survival function for the patient; and
providing a third result based on the parametric survival function and the survival time input.

2. The method of claim 1, wherein a risk factor of the plurality of risk factors comprises a categorical entry among the plurality of entries, and
wherein the method further comprises: in response to the input to select the cancer disease deep learning model,
converting the categorical entry to a numeric value; and
updating an entry of the plurality of entries, the entry corresponding to the categorical entry based on the of numeric value.

3. The method of claim 1,
wherein the plurality of entries corresponding to the plurality of risk factors comprises: an age of the patient, a cancer stage of the patient, an indication of regular aspirin usage, an indication of regular Ibuprofen usage, a number of first-degree relatives of the patient with any type of cancer, a sex of the patient, a current body mass index of the patient at baseline, a total number of years the patient smoked, an indication of whether the patient has ever gall bladder stones or inflammation, and an indication of whether the patient has ever high blood pressure.

4. The method of claim 1, wherein the trained cancer disease deep learning model uses three hyperbolic tangent (tanh) activation functions in three corresponding hidden layers.

5. The method of claim 1, wherein the trained cancer disease deep learning model uses a dropout, a batch-normalization, and an Adam optimizer.

6. The method of claim 1, wherein the plurality of patient inputs comprises: a race indication indicative of a race of the patient, an age group indication indicative of an age group of the patient, and a cancer stage indication indicative of a cancer stage of the patient.

7. The method of claim 6, wherein the plurality of patient inputs further comprises a gender indication indicative of a gender of the patient.

8. The method of claim 6, wherein the age group is one of a first age group between age 40 to age 59, a second age group between age 60 to age 79, and a third age group between age 80 and above,
wherein the race is one of Caucasian, African-American, and others, and
wherein the cancer stage is one of stage 1, stage 2, stage 3, and stage 4.

9. The method of claim 6, wherein the survival monitoring indicator for the patient is determined by:
$${SMI}_{jk}^{lm} = \frac{{n}_{jk}^{lm}}{{\sum }_{i = 1}^{n} log ( \frac{{( {t}_{n} )}_{jk}^{lm}}{{( {t}_{i} )}_{jk}^{lm}} )} ,$$
where l is the treatment option, k is the cancer stage indication, l is the age group indication, m is the race indication, SMIⱼₖ^{lm} is the survival monitoring indicator of the treatment (j) at the cancer stage (k) for the age group (l) belonging to the race (m), (tᵢ)ⱼₖ^{lm} is a longest time to death, and nⱼₖ^{lm} is a number of patients.

10. The method of claim 1, wherein the treatment option is one of chemotherapy, radiation, and a combination of chemotherapy and radiation.

11. The method of claim 1, wherein each of the first result, the second result, and the third result indicates at least one of: an increasing survival time of the patient, a decreasing survival time of the patient, or a same survival time of the patient.

12. The method of claim 11, further comprising:
determining a survival intensity function based on the survival monitoring indicator,
wherein in response to the survival intensity function decreasing with time, the second result indicates the increasing survival time of the patient,
wherein in response to the survival intensity function increasing with the time, the second result indicates the decreasing survival time of the patient, and
wherein in response to the survival intensity function being constant with time, the second result indicates the same survival time.

13. The method of claim 12, further comprising:
displaying a survival rate of changing of the patient based on the survival intensity function.

14. The method of claim 1, wherein the three parameters comprise a location parameter (\hat{k}), a scale parameter (\hat{δ}), and a shape parameter (\hat{μ}),
wherein the location parameter is determined by:
$$\hat{k} = \frac{{α}_{0} - 8 {α}_{1} - 9 {α}_{2}}{- {α}_{0} + {α}_{1} - {α}_{2}} ,$$
wherein the scale parameter is determined by:
$$\hat{σ} = \frac{( {α}_{0} - 2 {α}_{1} ) ( {α}_{0} - 3 {α}_{2} ) ( - 4 {α}_{1} + 6 {α}_{2} )}{{( - {α}_{0} + 4 {α}_{1} - 3 {α}_{2} )}^{2}} ,$$
wherein the shape parameter is determined by:
$$\hat{μ} = \frac{2 {α}_{0} {α}_{1} - 6 {α}_{0} {α}_{2} + 6 {α}_{1} {α}_{2}}{- {α}_{0} + 4 {α}_{1} - 3 {α}_{2}} ,$$
$$wherein {α}_{0} = ( μ + \frac{σ}{k} ) - \frac{σ}{k ( \frac{1}{k + 1} )} , \\ wherein {α}_{1} = \frac{1}{2 ( μ + \frac{σ}{k} )} - \frac{σ}{k ( \frac{1}{k + 2} )} , and \\ wherein {α}_{2} = \frac{1}{3 ( μ + \frac{σ}{k} )} - \frac{σ}{k ( \frac{1}{k + 3} )} .$$

15. The method of claim 4, wherein the parametric survival function (Ŝ) is determined by:
$$\hat{S} = [ 1 + the shape parameter {( \frac{\begin{matrix}the survival time \\ input - the \\ location parameter\end{matrix}}{the scale parameter} )}^{- 4.54} ] .$$

16. A method for cancer disease model training, comprising:
receiving a plurality of risk factors, each risk factor of the plurality of risk factors comprising a plurality of training entries;
normalizing the plurality of risk factors to produce a plurality of normalized risk factors;
identifying a combination of a plurality of hyper-parameters for a deep learning model, the combination producing a minimum root mean square error (RMSE) for the deep learning model among a plurality of combinations of the plurality of identified hyper-parameters; and
training the deep learning model with the plurality of normalized risk factors and the combination of the plurality of identified hyper-parameters, the deep learning model producing an estimated survival time.

17. The method of claim 16, wherein a risk factor of the plurality of risk factors comprises a plurality of categorical entries, the plurality of categorical entries corresponding to the plurality of training entries associated with the risk factor, and
wherein the method further comprises:
converting the plurality of categorical entries to a plurality of numeric values corresponding to the plurality of categorical entries; and
updating the risk factor based on the plurality of numeric values.

18. The method of claim 16, wherein the plurality of risk factors is normalized using Min-Max normalization being expressed as:
$$y *= \frac{y - min ( y )}{max ( y ) - min ( y )} ,$$
where y* is a normalized entry of a normalized risk factor of the plurality of normalized risk factors, y is a training entry of the plurality of training entries in a risk factor of the plurality of risk factors, max(y) is a maximum value among the plurality of entries in the risk factor, and min(y) is a minimum value among the plurality of training entries in the risk factor.

19. The method of claim 16, wherein the plurality of hyper-parameters comprises at least one of: an estimated time to train the deep learning model, a maximum depth of a tree of the deep learning model, a smallest number of instances in a child node of the deep learning model, a subsample ratio for regulating a number of samples provided to the tree of the deep learning model, or a column ratio when constructing the tree of the deep learning model.

20. A system for cancer disease estimation, comprising:
a memory storing a cancer disease deep learning model, a cancer disease stochastic model, and a cancer disease probability distribution function-based model;
a display; and
a processor communicatively coupled to the memory and display,
wherein the memory stores a set of instructions which, when executed by the processor, causes the processor to:
display on the display a user interface including a first indication corresponding to the cancer disease deep learning model, a second indication corresponding to the cancer disease stochastic model, and a third indication corresponding to the cancer disease probability distribution function-based model;
receive an input for selecting at least one of: the first indication, the second indication, or the third indication on the user interface;
in response to the input to select the first indication,
obtain a trained cancer disease deep learning model;
receive a plurality of entries corresponding to a plurality of risk factors for a patient;
transmit the plurality of entries to the trained cancer disease deep learning model; and
provide on the display a first result for the patient based on the trained cancer disease deep learning model;
in response to the input to select the second indication,
receive a plurality of patient inputs for the patient;
receive a treatment option;
determine a survival monitoring indicator for the patient based on the plurality of patient inputs and the treatment option; and
produce a second result based on the treatment option and the survival monitoring indication; and
in response to the input to select the third indication,
receive a survival time input for the patient;
determine three parameters;
determine a parametric survival function for the patient; and
provide a third result based on the parametric survival function and the survival time input.
